(19) United States
(12) Patent Application Publication
(10) Pub. No.: US 2020/0323823 A1
(43) Pub. Date: Oct. 15, 2020

(54) METHODS FOR THE TREATMENT OF DEPRESSION

(71) Applicant: Praxis Precision Medicines, Inc., Cambridge, MA (US)

(72) Inventors: Nicholas DEMARTINIS, Cambridge, MA (US); Carlos LOYA, Cambridge, MA (US)

(21) Appl. No.: 16/912,327

(22) Filed: Jun. 25, 2020

Related U.S. Application Data
(63) Continuation of application No. 16/716,252, filed on Dec. 16, 2019.
(60) Provisional application No. 62/927,396, filed on Oct. 29, 2019.
(60) Provisional application No. 62/813,493, filed on Mar. 4, 2019.
(60) Provisional application No. 62/779,895, filed on Dec. 14, 2018.

(51) Int. Cl. A61K 31/4164; A61K 31/137; A61K 47/06; A61K 31/42; A61K 31/5513

(52) U.S. Cl. CPC A61K 31/4164; A61K 31/137; A61K 31/5513; A61K 31/42; A61K 47/06

(57) ABSTRACT

The invention relates to methods of treating depression with Compound 1 or pharmaceutically acceptable salts thereof.

### CROSS-REFERENCE TO RELATED APPLICATIONS

This application is a continuation application of U.S. application Ser. No. 16/716,252, filed Dec. 16, 2019, which claims the benefit of priority to U.S. Application No. 62/927,396, filed Oct. 29, 2019, U.S. Provisional Application No. 62/813,493, filed Mar. 4, 2019, and U.S. Provisional Application No. 62/779,895, filed Dec. 14, 2018, which are hereby incorporated by reference in their entirety herein.

### FIELD OF THE DISCLOSURE

The present disclosure relates to methods for the treatment of depression using 3α-hydroxy-3β-methoxymethyl-21-(1′-imidazolyl)-5α-pregnan-20-one and salts thereof.

### BACKGROUND OF THE DISCLOSURE

3α-Hydroxy-3β-methoxymethyl-21-(1′-imidazolyl)-5α-pregnan-20-one (Compound 1) is a synthetic neuroactive steroid. Its primary molecular target is the γ-aminobutyric acid type A (GABA_{A}) receptor, where it acts as a positive allosteric modulator (PAM) of channel function. The structural formula of Compound 1 appears below.

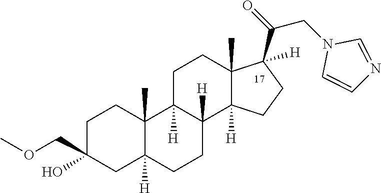

Neuroactive steroid GABA_{A} PAMs have demonstrated clinical efficacy in anesthesia, epilepsy, post-partum depression, and major depression.

### SUMMARY OF THE DISCLOSURE

The present disclosure, among other things, provides methods of treating depression by administering a therapeutically effective amount of Compound 1 or a pharmaceutically acceptable salt thereof to a patient in need thereof. In another aspect, the present invention provides methods of treating a mood or affective disorder selected from perimenopause, generalized anxiety disorder, panic disorder, social anxiety disorder, post-traumatic stress disorder, acute stress disorder, specific phobia, and selective mutism by administering a therapeutically effective amount of Compound 1 or a pharmaceutically acceptable salt thereof to a patient in need of thereof. In some embodiments, the present invention provides methods of treating acute stress disorder. In some embodiments, the present invention provides methods of treating post-traumatic stress disorder. In another aspect, the present invention provides methods of treating a substance abuse disorder by administering a therapeutically effective amount of Compound 1 or a pharmaceutically acceptable salt thereof to a patient in need of thereof. In some embodiments, the method comprises orally administering a daily dose of about 5 mg to about 120 mg of Compound 1 or a pharmaceutically acceptable salt thereof to a patient in need thereof.

In some embodiments, the patient in need of a treatment of depression is a patient with major depressive disorder (MDD). In certain embodiments, the patient has moderate MDD. In certain embodiments, the patient has severe MDD. In certain embodiments, the patient in need of a treatment of depression is a patient with MDD and insomnia. In certain embodiments, the patient in need of a treatment of depression is a patient with anxious MDD and insomnia. In certain embodiments, the patient in need of treatment of depression is a patient with MDD with anxious distress.

In some embodiments, the present disclosure provides adjunctive treatment for major depression comprising administering an effective amount of Compound 1 or a pharmaceutically acceptable salt thereof. In some embodiments, the patient in need of a treatment of depression is a patient that is partially responsive to other antidepressant therapies. In certain embodiments, the patient in need of a treatment of depression is a patient that is partially responsive to treatment with SSRIs. In some embodiments, the patient in need of a treatment of depression is a patient with depression is refractory to other therapies (i.e., treatment resistant depression).

In some embodiments, the present disclosure provides methods of treating depression by administering Compound 1 or a pharmaceutically acceptable salt thereof in combination with at least one additional antidepressant to a patient in need thereof. In certain embodiments, the additional antidepressant is selected from the group consisting of selective serotonin reuptake inhibitors, serotonin norepinephrine reuptake inhibitors, tricyclic antidepressants, monoamine oxidase inhibitors, mirtazapine, bupropion, lamotrigine and atypical antipsychotics.

### DETAILED DESCRIPTION OF THE DRAWINGS

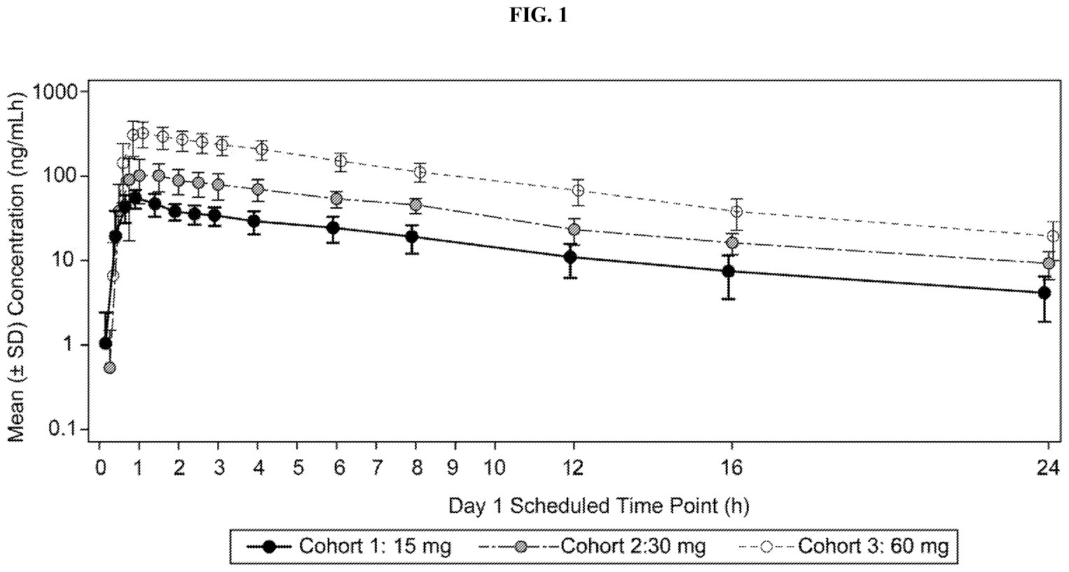

FIG. 1 is a graphical representation of the mean Compound 1 plasma concentration versus time for 15.0 mg daily administration of Compound 1 (Cohort 1), 30.0 mg daily administration of Compound 1 (Cohort 2), and a 60.0 mg daily administration of Compound 1 (Cohort 3).

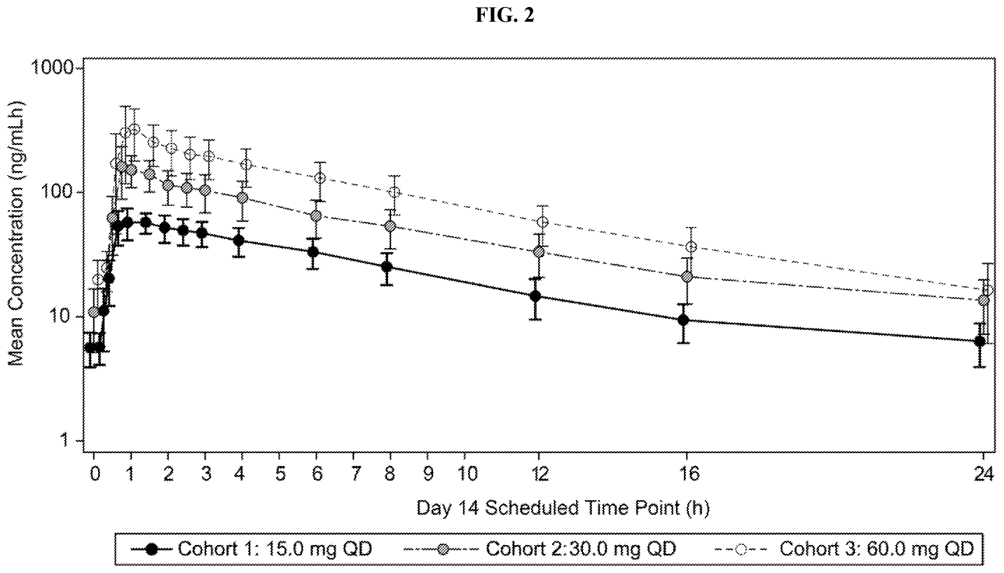

FIG. 2 is a graphical representation of the mean Compound 1 steady state plasma concentration versus time for 15.0 mg daily administration of Compound 1 (Cohort 1), 30.0 mg daily administration of Compound 1 (Cohort 2), and a 60.0 mg daily administration of Compound 1 (Cohort 3).

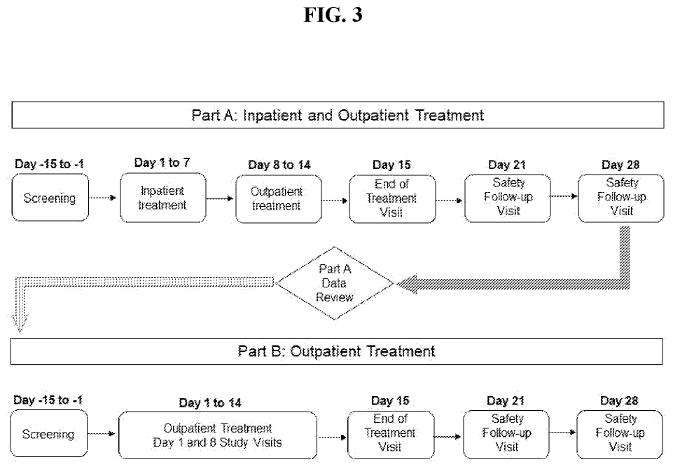

FIG. 3 is a graphical representation of the Phase 2 clinical study protocol described in Example 2.

### DEFINITIONS

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

The term “anxious distress” is used in this disclosure to mean the presence of at least two of the following symptoms during the majority of days of a major depressive episode or persistent depressive disorder (dysthymia): (1) Feeling keyed up or tense, (2) Feeling unusually restless, (3) Difficulty concentrating because of worry, (4) Fear that something awful may happen, and (5) Feeling that the individual might lose control of himself or herself. There are four severity categories of anxious distress: mild, moderate, moderate-severe and severe. “Mild anxious distress” is characterized by the presence of two of the five anxious distress symptoms. “Moderate anxious distress” is characterized by the presence of three of the five anxious distress symptoms. “Moderate-severe anxious distress” is characterized by the presence of four or five of the five anxious distress symptoms. “Moderate anxious distress” is characterized by the presence of four or five of the five anxious distress symptoms and with motor agitation.

The term “child and adolescent depression” is used in this disclosure to mean child and adolescent depression as defined in the Diagnostic and Statistical Manual of Mental Disorders (DSM-5).

The term “child and adolescent suicidal ideation and behavior” is used in this disclosure to mean a child and adolescent with suicidal ideation and behavior as assessed by the Columbia-suicide severity rating scale (C-SSRS) and defined in the Diagnostic and Statistical Manual of Mental Disorders (DSM-5).

The phrase “puberty” as used herein refers to puberty as defined by a validated staging system. In some embodiments, “puberty” refers to puberty as defined by the Stages of Reproductive Aging Workshop 10 Staging System (for female patients). In some embodiments, “puberty” refers to puberty as defined by the Tanner Stages Staging System.

The phrase “spermarche” and “spermarche transition” as used herein refers to spermarche and spermarche transition, respectively, as defined by a validated staging system. In some embodiments, “spermarche” and “spermarche transition” as used herein refers to spermarche and spermarche transition, respectively, as defined by the Tanner Stages Staging System.

The phrase “menarche” and “menarche transition” as used herein refers to menarche and menarche transition, respectively, as defined by a validated staging system. In some embodiments, “menarche” and “menarche transition” as used herein refers to menarche and menarche transition, respectively, as defined by the Stages of Reproductive Aging Workshop 10 Staging System. In some embodiments, “menarche” and “menarche transition” as used herein refers to menarche and menarche transition, respectively, as defined by the Tanner Stages Staging System.

The term “anxious major depressive disorder” (or anxious MDD) is used in this disclosure to mean a baseline 17-item Hamilton Depression Rating Scale (HAM-D) score ≥14 (excluding insomnia items) and a HAM-D anxiety/somatization score of ≥7.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “insomnia” is used in this disclosure to mean insomnia as defined in the Diagnostic and Statistical Manual of Mental Disorders (DSM-5).

The term “major depressive disorder” is used in this disclosure to mean major depressive disorder as defined in Diagnostic and Statistical Manual of Mental Disorders (DSM-5). The term “moderate major depressive disorder” is used in this disclosure to mean major depressive disorder where the number of symptoms, intensity of symptoms, and/or functional impairment are between those specified in the DSM-5 for “mild” and “severe.” The term “severe major depressive disorder” is used in this disclosure to mean major depressive disorder where the number of symptoms is substantially in excess of that required to make the diagnosis, the intensity of the symptoms is seriously distressing and unmanageable, and the symptoms markedly interfere with social and occupational functioning.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound, or a salt, solvate or ester thereof, that, when administered to a patient, is capable of performing the intended result. For example, an effective amount of a salt of Compound 1 is that amount that is required to reduce at least one symptom of depression in a patient. The actual amount that comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “perimenopause” as used herein refers to early and late menopause transition stages as well as the early postmenopause.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid.

The term “treating” as used herein with regard to a patient, refers to improving at least one symptom of the patient's disorder. Treating can be curing, improving, or at least partially ameliorating a disorder.

The term “therapeutic effect” as used herein refers to a desired or beneficial effect provided by the method and/or the composition. For example, the method for treating depression provides a therapeutic effect when the method reduces at least one symptom of depression in a patient.

### DETAILED DESCRIPTION OF THE DISCLOSURE

Major Depressive Disorder (MDD) is a common psychiatric illness that causes disability and diminishes quality of life, depletes limited health care resources, increases morbidity and mortality, and increases the rates of substance abuse and suicide. The incidence of MDD in the United States and Australia is about 7% (American Psychiatric Association. (2013). Diagnostic and statistical manual of mental disorders (5th Ed.). Arlington, Va.: American Psychiatric Publishing) and 5% (Australian Bureau of Statistics. (2008). National Survey of Mental Health and Wellbeing: Summary of results, 2007, cat no. 4326.0. Canberra: ABS), respectively. Current treatment options for MDD patients are limited and include serotonin reuptake inhibitors (SSRIs) and serotonin norepinephrine uptake inhibitors (SNRIs). However, SSRIs and SNRIs have serious liabilities in the context of treating MDD, such as a substantial delay in the onset of efficacy (several weeks) and high rates of treatment failure, e.g., about 33% of MDD patients fail to achieve full symptomatic remission despite multiple treatment regimens (Rush A J, et al. Acute and longer-term outcomes in depressed outpatients requiring one or several treatment steps: a STAR*D report. Am J Psychiatry. 2006; 163:1905-1917.).

Disturbances of the GABAergic system have been implicated in the development of depression and anxiety. A growing body of preclinical and clinical evidence supports the hypothesis that GABA_{A} hypofunction plays a role in the pathophysiology of depression and anxiety (Luscher B, Shen Q, Sahir N. The GABAergic deficit hypothesis of major depressive disorder. Mol Psychiatry. 2011; 16 (4):383-406). Supporting this hypothesis, drugs that enhance GABAergic function have shown some clinical benefit in the treatment of mood disorders. For example, benzodiazepines, which are positive allosteric modulators (PAMs) of the GABA_{A} receptor, are highly efficacious in the treatment of anxiety disorders. However, due to lack of efficacy, significant side effects (e.g., sedation), tolerance development, withdrawal symptoms upon cessation and a significant abuse potential, benzodiazepines are not recommended for the treatment of MDD. Therefore, there is a need for new treatment options for patients suffering from depression, such as MDD.

Neuroactive steroids (NASs) are a family of compounds (synthetic and naturally occurring) that affect neurophysiological functions through allosteric modulation of GABA_{A} receptors. The endogenous NASs allopregnanolone and pregnanolone are GABA_{A} PAMs that are dysregulated in mood disorders and show preclinical efficacy in animal models of anxiety and depression. NASs bind to a different binding site on the GABA_{A} receptor than benzodiazepines or the endogenous agonist GABA (Hosie A M, Wilkins M E, Da Silva H M A, Smart T G. Endogenous neurosteroids regulate GABA_{A} receptors through two discrete transmembrane sites. Nature. 2006; 444(7118):486-489.). Benzodiazepines exclusively potentiate GABA_{A} receptors that contain a gamma subunit, which are primarily localized at synapses. In contrast, NASs bind to alpha and beta subunits, which are present in a larger proportion of GABA_{A} receptors, resulting broad activity at both synaptic and extrasynaptic sites. This differentiating pharmacology supports the utility of NAS for indications where benzodiazepines have not exhibited significant utility, such as MDD.

In one aspect, the present invention provides a method of treating depression comprising administering an effective amount of Compound 1 or a pharmaceutically acceptable salt thereof to a patient in need of such treatment. In another aspect, the present invention provides a method of treating a mood or affective disorder selected from perimenopause, generalized anxiety disorder, panic disorder, social anxiety disorder, acute stress disorder, post-traumatic stress disorder, specific phobia, and selective mutism comprising administering an effective amount of Compound 1 or a pharmaceutically acceptable salt thereof to a patient in need of such treatment. In accordance with some embodiments of the present invention, at least about 15 mg, about 30 mg, about 45 mg, about 60 mg, about 75 mg, about 80 mg, about 85 mg, about 90 mg, about 95 mg, about 100 mg, about 105 mg, about 110 mg, about 115 mg or about 120 mg of Compound 1 or a pharmaceutically acceptable salt thereof is administered.

### Compound 1

Compound 1 as employed in the present methods can form a part of a pharmaceutical composition by combining Compound 1, or a pharmaceutically acceptable salt thereof, with a pharmaceutically acceptable carrier. Additionally, the compositions can include an additive selected from the group consisting of adjuvants, excipients, diluents, release-modifying agents and stabilizers. The composition can be an immediate release formulation, a delayed release formulation, a sustained release formulation or an extended release formulation.

3α-Hydroxy-3β-methoxymethyl-21-(1′-imidazolyl)-5α-pregnan-20-one (Compound 1) is a synthetic neuroactive steroid. The structural formula of Compound 1 appears below.

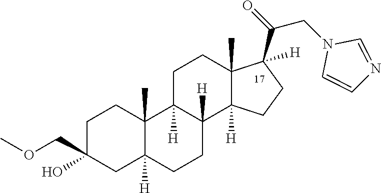

Compound 1 is a neuroactive steroid GABA-A positive allosteric modulator (PAM) with high potency similar to clinical stage neuroactive steroids (allopregnanolone, ganaxolone, SAGE-217, alphaxolone).

The synthesis of Compound 1 is described in U.S. Publication Nos. 2004/034002 and 2009/0118248; crystalline polymorph of Compound 1 free base is described in U.S. Publication No. 2006/0074059; pharmaceutical compositions containing Compound 1 are described in U.S. Publication No. 2009/0131383, which are hereby incorporated by reference in their entirety for all purposes.

In some embodiments, the Compound 1 used in the formulations and methods of the present disclosure is a pharmaceutically acceptable salt of Compound 1. Salts of Compound 1 and polymorphs thereof are described in U.S. application Ser. No. 16/517,369, which is hereby incorporated by reference in its entirety. In some embodiments, the pharmaceutically acceptable salt of Compound 1 used in the formulations and methods of the present disclosure is selected from the group consisting of hydrobromide, citrate, malate, mesylate, phosphate, tartrate, hydrochloride, tosylate, glucuronate, ethanesulfonate, fumarate, sulfate, napthalene-2-sulfonate, ascorbate, oxalate, napthalene-1,5-disulfonate, malonate, aminosalicylate, benzenesulfonate, isethionate, gentistate, 1-hydroxy-2-napthoate, dichloroacetate, cyclamate, and ethane-1,2-disulfonate salts. In certain embodiments, the salt of Compound 1 is Compound 1 Hydrobromide. In certain embodiments, the salt of Compound 1 is Compound 1 Citrate. In certain embodiments, the salt of Compound 1 is Compound 1 L-Malate. In certain embodiments, the salt of Compound 1 is Compound 1 Mesylate. In certain embodiments, the salt of Compound 1 is Compound 1 Phosphate. In certain embodiments, the salt of Compound 1 is Compound 1 L(+)-Tartrate. In certain embodiments, the salt of Compound 1 is Compound 1 Hydrochloride. In certain embodiments, the salt of Compound 1 is Compound 1 Tosylate. In certain embodiments, the salt of Compound 1 is Compound 1 Glucuronate. In certain embodiments, the salt of Compound 1 is Compound 1 Ethanesulfonate.

Formulations

The methods of the present invention can employ various formulations for administration to patients, e.g., humans in unit dosage forms, such as tablets, capsules, pills, powders, granules, sterile parenteral solutions or suspensions (e.g., intramuscular (IM), subcutaneous (SC) and intravenous (IV)), transdermal patches, and oral solutions or suspensions, and oil-water emulsions containing suitable quantities of Compound 1 or a pharmaceutically acceptable salt thereof.

Oral pharmaceutical dosage forms can be either solid or liquid. The solid dosage forms can be tablets, capsules, granules, films (e.g., buccal films) and bulk powders. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art. In some embodiments, the present oral dosage forms may include orally disintegrating tablets.

Pharmaceutically acceptable carriers utilized in tablets include binders, lubricants, diluents, disintegrating agents, coloring agents, flavoring agents, and wetting agents.

Liquid oral dosage forms include aqueous solutions, emulsions, suspensions, solutions and/or suspensions reconstituted from non-effervescent granules and effervescent preparations reconstituted from effervescent granules.

Aqueous solutions include, for example, elixirs and syrups. Emulsions can be either oil-in water or water-in-oil. Elixirs are clear, sweetened, hydroalcoholic preparations. Pharmaceutically acceptable carriers used in elixirs include solvents. Syrups can be concentrated aqueous solutions of a sugar, for example, sucrose, and can contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions can use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substance used in effervescent granules, to be reconstituted into a liquid oral dosage form, can include organic acids and a source of carbon dioxide. Coloring and flavoring agents can be used in all of the above dosage forms.

In some embodiments, the present disclosure provides a pharmaceutical composition comprising a salt of Compound 1. In some embodiments, the salt of Compound 1 is Compound 1 Hydrobromide, Compound 1 Citrate, Compound 1 L-Malate, Compound 1 Mesylate, Compound 1 Phosphate, Compound 1 L(+)-Tartrate, Compound 1 Hydrochloride, Compound 1 Tosylate, Compound 1 Glucuronate, or Compound 1 Ethanesulfonate.

Co-Therapy

While the compositions can be administered as the sole active pharmaceutical ingredient (i.e., Compound 1) or sole active anti-depressant ingredient in the methods described herein, in some embodiments they can also be used in combination with one or more ingredients which are known to be therapeutically effective against depression and/or compliment the antidepressant effect of the Compound 1 ingredient.

For example, in some embodiments, the present methods can employ Compound 1, or a pharmaceutically acceptable salt thereof, in conjunction with one or more additional anti-antidepressants agents.

In some embodiments, Compound 1 or a pharmaceutically acceptable salt thereof is administered in combination with an additional anti-depressant agent, e.g., co-formulated or administered separately. In some embodiments, Compound 1 or a pharmaceutically acceptable salt thereof is administered in conjunction with one or more selective serotonin reuptake inhibitors, serotonin norepinephrine reuptake inhibitors, tricyclic antidepressants, monoamine oxidase inhibitors, mirtazapine, bupropion, lamotrigine atypical antipsychotics, or combinations thereof. In some embodiments, Compound 1 or a pharmaceutically acceptable salt thereof is administered in combination with electroconvulsive therapy (ECT). In some embodiments, Compound 1 or a pharmaceutically acceptable salt thereof is administered in combination with transcranial magnetic stimulation (TMS).

In some embodiments, Compound 1 or a pharmaceutically acceptable salt thereof is administered in conjunction with one or more selective serotonin reuptake inhibitors. In certain embodiments, the one or more selective serotonin reuptake inhibitors is selected from the group consisting of fluoxetine, escitalopram, citalopram, sertraline, and paroxetine.

In some embodiments, Compound 1 or a pharmaceutically acceptable salt thereof is administered in conjunction with one or more serotonin norepinephrine reuptake inhibitors. In certain embodiments, the one or more serotonin norepinephrine reuptake inhibitors is selected from the group consisting of venlafaxine and duloxetine.

In some embodiments, Compound 1 or a pharmaceutically acceptable salt thereof is administered in conjunction with one or more tricyclic antidepressants. In certain embodiments, the one or more tricyclic antidepressants is selected from the group consisting of amitriptyline, imipramine, and nortriptyline.

In some embodiments, Compound 1 or a pharmaceutically acceptable salt thereof is administered in conjunction with one or more monoamine oxidase inhibitors. In certain embodiments, the one or more monoamine oxidase inhibitors is selected from the group consisting of phenelzine and tranylcypromine.

In some embodiments, Compound 1 or a pharmaceutically acceptable salt thereof is administered in conjunction with one or more atypical antipsychotics. In certain embodiments, the one or more atypical antipsychotics is selected from the group consisting of lurasidone, aripiprazole, risperidone, olanzapine, quetiapine, ziprasidone, clozapine, iloperidone, paliperidone, asenapine and olanzapine/fluoxetine.

Dosing

The invention provides methods for treating depression by administering an effective amount of Compound 1 or a pharmaceutically acceptable salt thereof to a patient in need thereof. An effective amount is an amount sufficient to eliminate or significantly reduce depression symptoms or to alleviate those symptoms (e.g., reduce the symptoms, such as depressed mood, compared to the symptoms present prior to treatment).

According to some embodiments of the present invention, administering Compound 1 or a pharmaceutically acceptable salt thereof provides statistically significant therapeutic effect. In one embodiment, the statistically significant therapeutic effect is determined based on one or more standards or criteria provided by one or more regulatory agencies in the United States, e.g., FDA or other countries (such as Australia). In some embodiments, the statistically significant therapeutic effect is determined based on results obtained from regulatory agency approved clinical trial set up and/or procedure.

In some embodiments, the statistically significant therapeutic effect is determined based on a patient population of at least 20, 50, 60, 100, 200, 300, 400, 500, 600, 700, 800, 900, 1000 or 2000. In some embodiments, the statistically significant therapeutic effect is determined based on data obtained from randomized and double-blinded clinical trial set up. In some embodiments, the statistically significant therapeutic effect is determined based on data with a p value of less than or equal to about 0.05, 0.04, 0.03, 0.02 or 0.01. In some embodiments, the statistically significant therapeutic effect is determined based on data with a confidence interval greater than or equal to 95%, 96%, 97%, 98% or 99%.

In some embodiments, the statistically significant therapeutic effect is determined by a randomized double-blind clinical trial of patients treated with Compound 1 or a pharmaceutically acceptable salt thereof and optionally in combination with standard care. In some embodiments, the statistically significant therapeutic effect is determined by a randomized clinical trial and using Hamilton Depression Rating Scale (HAM-D) as primary efficacy parameter and optionally in combination with any other commonly accepted criteria for depression assessment.

In general, statistical analysis can include any suitable method permitted by a regulatory agency, e.g., FDA in the US or Europe or any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM) and Cox regression analysis.

According to the present invention, Compound 1 is administered on a once or twice a day basis to provide effective relief of the symptoms of depression (for example, major depressive disorder, major depressive disorder with suicidal risk, clinical depression, postnatal or postpartum depression, treatment resistant postpartum depression, perimenopausal depression, child and adolescent depression, premenstrual dysphoric disorder (PMDD), atypical depression, melancholic depression, Psychotic Major Depression (PMD), catatonic depression, Seasonal Affective Disorder (SAD), persistent depressive disorder (dysthymia), double depression, Depressive Personality Disorder (DPD), Recurrent Brief Depression (RBD), minor depressive disorder, bipolar disorder or manic depressive disorder, bipolar depression with suicidal risk, post-traumatic stress disorders, depression caused by chronic medical conditions, depressive disorder due to another medical condition, treatment-resistant depression, refractory depression, substance/medication induced depressive disorder, depression with anxiety, suicidality, suicidal ideation, or suicidal behavior) or a mood or affective disorder selected from perimenopause, generalized anxiety disorder, panic disorder, social anxiety disorder, acute stress disorder, post-traumatic stress disorder, specific phobia, and selective mutism. In some embodiments, Compound 1 is administered on a once or twice a day basis to provide effective relief of the symptoms of acute stress disorder. In some embodiments, Compound 1 is administered on a once or twice a day basis to provide effective relief of the symptoms of post-traumatic stress disorder.

In some embodiments, a total daily dose is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg.

In certain embodiments, the total daily dose of Compound 1 is from about 5 mg to about 120 mg, including about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg, including all ranges there between. In certain embodiments, the total daily dose of Compound 1 is from about 15 mg to about 60 mg. In certain embodiments, the total daily dose of Compound 1 is from about 15 mg to about 80 mg. In certain embodiments, the total daily dose of Compound 1 is from about 15 mg to about 100 mg. In certain embodiments, the total daily dose of Compound 1 is from about 45 mg to about 60 mg. In certain embodiments, the total daily dose of Compound 1 is from about 45 mg to about 80 mg.

In the embodiments described herein, reference is made to the dose of Compound 1 for the treatment of depression (which includes acute treatment of depression and chronic treatment of depression). However, the present disclosure contemplates the disclosed doses for the treatment of a mood or affective disorder selected from perimenopause, generalized anxiety disorder, panic disorder, social anxiety disorder, acute stress disorder, post-traumatic stress disorder, specific phobia, and selective mutism.

In some embodiments, the total daily dose of Compound 1 is at least about 5 mg a day for the treatment of depression. In some embodiments, the total daily dose of Compound 1 is at least about 10 mg a day for the treatment of depression. In some embodiments, the total daily dose of Compound 1 is at least about 15 mg a day for the treatment of depression. In some embodiments, the total daily dose of Compound 1 is at least about 20 mg a day for the treatment of depression. In some embodiments, the total daily dose of Compound 1 is at least about 25 mg a day for the treatment of depression. In some embodiments, the total daily dose of Compound 1 is at least about 30 mg a day for the treatment of depression. In some embodiments, the total daily dose of Compound 1 is at least about 35 mg a day for the treatment of depression. In some embodiments, the total daily dose of Compound 1 is at least about 40 mg a day for the treatment of depression. In some embodiments, the total daily dose of Compound 1 is at least about 45 mg a day for the treatment of depression. In some embodiments, the total daily dose of Compound 1 is at least about 50 mg a day for the treatment of depression. In some embodiments, the total daily dose of Compound 1 is at least about 55 mg a day for the treatment of depression. In some embodiments, the total daily dose of Compound 1 is at least about 60 mg a day for the treatment of depression. In some embodiments, the total daily dose of Compound 1 is at least about 65 mg a day for the treatment of depression. In some embodiments, the total daily dose of Compound 1 is at least about 70 mg a day for the treatment of depression. In some embodiments, the total daily dose of Compound 1 is at least about 75 mg a day for the treatment of depression. In some embodiments, the total daily dose of Compound 1 is at least about 80 mg a day for the treatment of depression. In some embodiments, the total daily dose of Compound 1 is at least about 85 mg a day for the treatment of depression. In some embodiments, the total daily dose of Compound 1 is at least about 90 mg a day for the treatment of depression. In some embodiments, the total daily dose of Compound 1 is at least about 95 mg a day for the treatment of depression. In some embodiments, the total daily dose of Compound 1 is at least about 100 mg a day for the treatment of depression. In some embodiments, the total daily dose of Compound 1 is at least about 105 mg a day for the treatment of depression. In some embodiments, the total daily dose of Compound 1 is at least about 110 mg a day for the treatment of depression. In some embodiments, the total daily dose of Compound 1 is at least about 115 mg a day for the treatment of depression. In some embodiments, the total daily dose of Compound 1 is at least about 120 mg a day for the treatment of depression.

In some embodiments, the total daily dose of Compound 1 is about 5 mg a day for the treatment of depression. In some embodiments, the total daily dose of Compound 1 is about 10 mg a day for the treatment of depression. In some embodiments, the total daily dose of Compound 1 is about 15 mg a day for the treatment of depression. In some embodiments, the total daily dose of Compound 1 is about 20 mg a day for the treatment of depression. In some embodiments, the total daily dose of Compound 1 is about 25 mg a day for the treatment of depression. In some embodiments, the total daily dose of Compound 1 is about 30 mg a day for the treatment of depression. In some embodiments, the total daily dose of Compound 1 is about 35 mg a day for the treatment of depression. In some embodiments, the total daily dose of Compound 1 is about 40 mg a day for the treatment of depression. In some embodiments, the total daily dose of Compound 1 is about 45 mg a day for the treatment of depression. In some embodiments, the total daily dose of Compound 1 is about 50 mg a day for the treatment of depression. In some embodiments, the total daily dose of Compound 1 is about 55 mg a day for the treatment of depression. In some embodiments, the total daily dose of Compound 1 is about 60 mg a day for the treatment of depression. In some embodiments, the total daily dose of Compound 1 is about 65 mg a day for the treatment of depression. In some embodiments, the total daily dose of Compound 1 is about 70 mg a day for the treatment of depression. In some embodiments, the total daily dose of Compound 1 is about 75 mg a day for the treatment of depression. In some embodiments, the total daily dose of Compound 1 is about 80 mg a day for the treatment of depression. In some embodiments, the total daily dose of Compound 1 is about 85 mg a day for the treatment of depression. In some embodiments, the total daily dose of Compound 1 is about 90 mg a day for the treatment of depression. In some embodiments, the total daily dose of Compound 1 is about 95 mg a day for the treatment of depression. In some embodiments, the total daily dose of Compound 1 is about 100 mg a day for the treatment of depression. In some embodiments, the total daily dose of Compound 1 is about 105 mg a day for the treatment of depression. In some embodiments, the total daily dose of Compound 1 is about 110 mg a day for the treatment of depression. In some embodiments, the total daily dose of Compound 1 is about 115 mg a day for the treatment of depression. In some embodiments, the total daily dose of Compound 1 is about 120 mg a day for the treatment of depression.

In some embodiments, about 5 mg of Compound 1 once a day is selected to provide a substantial reduction in depression. In some embodiments, about 5 mg of Compound 1 twice a day is selected to provide a substantial reduction in depression. In some embodiments, about 10 mg of Compound 1 once a day is selected to provide a substantial reduction in depression. In some embodiments, about 10 mg of Compound 1 twice a day is selected to provide a substantial reduction in depression. In some embodiments, about 15 mg of Compound 1 once a day is selected to provide a substantial reduction in depression. In some embodiments, about 15 mg of Compound 1 twice a day is selected to provide a substantial reduction in depression. In some embodiments, about 20 mg of Compound 1 once a day is selected to provide a substantial reduction in depression. In some embodiments, about 20 mg of Compound 1 twice a day is selected to provide a substantial reduction in depression. In some embodiments, about 25 mg of Compound 1 once a day is selected to provide a substantial reduction in depression. In some embodiments, about 25 mg of Compound 1 twice a day is selected to provide a substantial reduction in depression. In some embodiments, about 30 mg of Compound 1 once a day is selected to provide a substantial reduction in depression. In some embodiments, about 30 mg of Compound 1 twice a day is selected to provide a substantial reduction in depression. In some embodiments, about 30 mg of Compound 1 once a day is selected to provide a substantial reduction in depression. In some embodiments, about 30 mg of Compound 1 twice a day is selected to provide a substantial reduction in depression. In some embodiments, about 35 mg of Compound 1 once a day is selected to provide a substantial reduction in depression. In some embodiments, about 35 mg of Compound 1 twice a day is selected to provide a substantial reduction in depression. In some embodiments, about 40 mg of Compound 1 once a day is selected to provide a substantial reduction in depression. In some embodiments, about 40 mg of Compound 1 twice a day is selected to provide a substantial reduction in depression. In some embodiments, about 45 mg of Compound 1 once a day is selected to provide a substantial reduction in depression. In some embodiments, about 45 mg of Compound 1 twice a day is selected to provide a substantial reduction in depression. In some embodiments, about 50 mg of Compound 1 once a day is selected to provide a substantial reduction in depression. In some embodiments, about 50 mg of Compound 1 twice a day is selected to provide a substantial reduction in depression. In some embodiments, about 55 mg of Compound 1 once a day is selected to provide a substantial reduction in depression. In some embodiments, about 55 mg of Compound 1 twice a day is selected to provide a substantial reduction in depression. In some embodiments, about 60 mg of Compound 1 once a day is selected to provide a substantial reduction in depression. In some embodiments, about 60 mg of Compound 1 twice a day is selected to provide a substantial reduction in depression. In some embodiments, about 65 mg of Compound 1 once a day is selected to provide a substantial reduction in depression. In some embodiments, about 70 mg of Compound 1 once a day is selected to provide a substantial reduction in depression. In some embodiments, about 75 mg of Compound 1 once a day is selected to provide a substantial reduction in depression. In some embodiments, about 80 mg of Compound 1 once a day is selected to provide a substantial reduction in depression. In some embodiments, about 85 mg of Compound 1 once a day is selected to provide a substantial reduction in depression. In some embodiments, about 90 mg of Compound 1 once a day is selected to provide a substantial reduction in depression. In some embodiments, about 95 mg of Compound 1 once a day is selected to provide a substantial reduction in depression. In some embodiments, about 100 mg of Compound 1 once a day is selected to provide a substantial reduction in depression. In some embodiments, about 105 mg of Compound 1 once a day is selected to provide a substantial reduction in depression. In some embodiments, about 110 mg of Compound 1 once a day is selected to provide a substantial reduction in depression. In some embodiments, about 115 mg of Compound 1 once a day is selected to provide a substantial reduction in depression. In some embodiments, about 120 mg of Compound 1 once a day is selected to provide a substantial reduction in depression.

Reduction of depression in patients with depressive conditions can be determined by various methods. In some embodiments, the effectiveness of a dosage regimen can be determined by evaluation via Hamilton Depression Rating Scale (HAM-D). In some embodiments, the effectiveness of a dosage regimen can be determined by evaluation via a Montgomery Asberg Depression Rating Scale (MADRS). In some embodiments, the effectiveness of a dosage regimen can be determined by evaluation via HAM-D, MADRS, Hamilton Rating Scale for anxiety (HAM-A), Clinical Global Impression (CGI) subscale scores (i.e., Severity of Illness Subscale (CGI-S), Global Improvement Subscale (CGI-I), or Efficacy Index Subscale), Symptoms of Depression Questionnaire (SDQ), Pittsburgh Sleep Quality Index (PSQI), or any combination thereof.

In some embodiments, the effectiveness of a dosage regimen can be determined by evaluation via a total HAM-D value as a primary efficacy endpoint in association with secondary efficacy endpoints such as the MADRS, HAM-A, CGI-S, CGI-I, SDQ, PSQI, or any combination thereof.

According to some embodiments of the present invention, the dosing frequency and dose amount per administration of Compound 1 are selected to provide therapeutic effects for the treatment of depression selected from major depressive disorder, major depressive disorder with suicidal risk, clinical depression, postnatal or postpartum depression, treatment resistant postpartum depression, perimenopausal depression, premenstrual dysphoric disorder (PMDD), atypical depression, melancholic depression, Psychotic Major Depression (PMD), catatonic depression, Seasonal Affective Disorder (SAD), persistent depressive disorder (dysthymia), double depression, Depressive Personality Disorder (DPD), Recurrent Brief Depression (RBD), minor depressive disorder, bipolar disorder or manic depressive disorder, bipolar depression with suicidal risk, post-traumatic stress disorders, depression caused by chronic medical conditions, depressive disorder due to another medical condition, treatment-resistant depression, refractory depression, substance/medication induced depressive disorder, depression with anxiety, suicidality, suicidal ideation, or suicidal behavior.

According to some embodiments of the present invention, the dosing frequency and dose amount per administration of Compound 1 are selected to provide therapeutic effects for the treatment of major depressive disorder. In certain embodiments, the dosing frequency and amount per administration of Compound 1 are selected to provide for the treatment of moderate major depressive disorder. In certain embodiments, the dosing frequency and amount per administration of Compound 1 are selected to provide for the treatment of severe major depressive disorder. In certain embodiments, the dosing frequency and amount per administration of Compound 1 are selected to provide for the treatment of severe major depressive disorder in a patient having a total HAM-D value of at least 22.

According to some embodiments of the present disclosure, the dosing frequency and dose amount per administration of Compound 1 are selected to provide therapeutic effects for the treatment of child and adolescent depression. In some embodiments, the dosing frequency and dose amount per administration of Compound 1 are selected to provide therapeutic effects for the treatment of child and adolescent depression during puberty. In some embodiments, the dosing frequency and dose amount per administration of Compound 1 are selected to provide therapeutic effects for the treatment of child and adolescent depression during menarche or menarche transition. In some embodiments, the dosing frequency and dose amount per administration of Compound 1 are selected to provide therapeutic effects for the treatment of child and adolescent depression during spermarche or spermarche transition.

According to some embodiments of the present invention, the dosing frequency and dose amount per administration of Compound 1 are selected to provide therapeutic effects for the treatment of depression that is refractory to other treatments (i.e., treatment resistant depression).

According to some embodiments of the present invention, the dosing frequency and dose amount per administration of Compound 1 are selected to provide therapeutic effects for the treatment of depression that is partially responsive to other antidepressant therapies. According to some embodiments of the present invention, the dosing frequency and dose amount per administration of Compound 1 are selected to provide an adjunctive treatment for major depression. According to some embodiments of the present invention, the dosing frequency and dose amount per administration of Compound 1 are selected to provide therapeutic effects for the treatment of depression that is partially responsive to treatment with SSRIs.

In certain embodiments, the dosing frequency and dose amount per administration of Compound 1 are selected to provide therapeutic effects for the treatment of MDD that is partially responsive to other antidepressant therapies. In certain embodiments, the MDD patient that is partially responsive to other antidepressant therapies is an adult patient meeting DSM-IV criteria for MDD who had had an inadequate response to prior antidepressant therapy (1 to 3 courses) in the current episode and who had demonstrated an inadequate response to 8 weeks of prospective antidepressant therapy. Inadequate response for prospective treatment is defined as less than 50% improvement on the 17-item version of the Hamilton Depression Rating Scale (HAM-D), minimal HAM-D score of 14, and a Clinical Global Impressions Improvement rating of no better than minimal improvement. Inadequate response to prior treatment is defined as less than 50% improvement as perceived by the patient after a minimum of 6 weeks of antidepressant therapy at or above the minimal effective dose. In certain embodiments, the dosing frequency and dose amount per administration of Compound 1 are selected to provide therapeutic effects for the treatment of moderate MDD that is partially responsive to other antidepressant therapies. In certain embodiments, the dosing frequency and dose amount per administration of Compound 1 are selected to provide therapeutic effects for the treatment of severe MDD that is partially responsive to other antidepressant therapies. In certain embodiments, the dosing frequency and dose amount per administration of Compound 1 are selected to provide therapeutic effects for the treatment of MDD that is partially responsive to treatment with SSRIs. In certain embodiments, the dosing frequency and dose amount per administration of Compound 1 are selected to provide therapeutic effects for the treatment of moderate MDD that is partially responsive to treatment with SSRIs. In certain embodiments, the dosing frequency and dose amount per administration of Compound 1 are selected to provide therapeutic effects for the treatment of severe MDD that is partially responsive to treatment with SSRIs.

In certain embodiments, the dosing frequency and dose amount per administration of Compound 1 are selected to provide therapeutic effects for the treatment of MDD in a patient with insomnia. In certain embodiments, the dosing frequency and dose amount per administration of Compound 1 are selected to provide therapeutic effects for the treatment of MDD in a patient with insomnia characterized by difficulties with sleep initiation. In certain embodiments, the dosing frequency and dose amount per administration of Compound 1 are selected to provide therapeutic effects for the treatment of MDD and the treatment of insomnia in an MDD patient with insomnia. In certain embodiments, the dosing frequency and dose amount per administration of Compound 1 are selected to provide therapeutic effects for the treatment of MDD and the treatment of insomnia in an anxious MDD patient with insomnia.

In certain embodiments, after treatment the patient experiences a substantial reduction of MDD and insomnia compared to prior to the treatment. In certain embodiments, after treatment for at least one week the patient experiences a substantial reduction of MDD and insomnia compared to prior to the treatment. According to this embodiment, the substantial reduction in insomnia may be expressed using any of the methods described herein (for example, an improvement in polysomnography parameters, such as a decline in latency to persistent sleep (LPS) compared to prior to the treatment, decline in wake time after sleep onset (WASO) compared to prior to the treatment, etc.) and the substantial reduction in MDD may be expressed using any of the methods described herein (for example, decline in total Hamilton Depression Rating Scale (HAM-D) value compared to prior to the treatment, improvement in the Montgomery Asberg Depression Rating Scale value compared to prior to the treatment, etc.).

The sleep parameters described herein (including wake time after sleep onset, total sleep time, sleep efficiency and latency to persistent sleep) may be measured by polysomnography using methods that are known to those skilled in the art.

Wake time after sleep onset is the wakefulness time occurring after defined sleep onset. In some embodiments, after the treatment the patient experiences a substantial reduction of insomnia that is characterized by at least about a 30% decline in wake time after sleep onset (WASO) compared to prior to the treatment. In some embodiments, the reduction of insomnia is characterized by a decline in WASO ranging from about 30% to about 100%, for example, about 30%, about 40%, about 50%, about 60%, about 70%, about 80%, about 90%, and about 100%, compared to prior to the treatment.

Total Sleep Time is the amount of actual sleep time in a sleep episode, i.e., the total sleep episode less the awake time. In some embodiments, after the treatment the patient experiences a substantial reduction of insomnia that is characterized by at least about a 30% increase in Total Sleep Time (TST) compared to prior to the treatment. In some embodiments, the reduction of insomnia is characterized by an increase in TST ranging from about 30% to about 100%, for example, about 30%, about 40%, about 50%, about 60%, about 70%, about 80%, about 90%, and about 100%, compared to prior to the treatment.

Sleep efficiency is the percentage of total time in bed actually spend in sleep. An increase in sleep efficiency correlates to an improvement in insomnia. In some embodiments, after the treatment the patient experiences a substantial reduction of insomnia that is characterized by at least about a 30% increase in sleep efficiency (SE) compared to prior to the treatment. In some embodiments, the reduction of insomnia is characterized by an increase in SE value ranging from about 30% to about 100%, for example, about 30%, about 40%, about 50%, about 60%, about 70%, about 80%, about 90%, and about 100%, compared to prior to the treatment.

Latency to persistent sleep is the length of time that it takes to accomplish the transition from full wakefulness to sleep. In some embodiments, after the treatment the patient experiences a substantial reduction of insomnia that is characterized by at least about a 30% decrease in latency to persistent sleep (LPS) compared to prior to the treatment. In some embodiments, the reduction of insomnia is characterized by a decline in LPS value ranging from about 30% to about 100%, for example, about 30%, about 40%, about 50%, about 60%, about 70%, about 80%, about 90%, and about 100%, compared to prior to the treatment.

The Pittsburgh Sleep Quality Index (PSQI) is a 19-item self-report scale that assesses sleep quality and disturbances for the month preceding the assessment (Buysse D. J., The Pittsburgh Sleep Quality Index: a New Instrument for Psychiatric Practice and Research. Psychiatry Res. 1989 May; 28(2), pages 193-213.). The scale generates seven “component” scores that differentiate “poor” from “good” sleep quality: subjective sleep quality, sleep latency, sleep duration, habitual sleep efficiency, sleep disturbances, use of sleeping medication, and daytime dysfunction. The sum of scores for these seven components yields the Global PSQI score. A Global PSQI score of “5” or greater indicates poor sleep quality. In some embodiments, after the treatment the patient experiences a substantial reduction of insomnia that is characterized by at least a one point decline in Global Pittsburgh Sleep Quality Index (PSQI) score compared to prior to the treatment. In some embodiments, the reduction of insomnia is characterized by a one point decline in Global PSQI score compared to prior to the treatment. In some embodiments, the reduction of insomnia is characterized by a two point decline in Global PSQI score compared to prior to the treatment. In some embodiments, the reduction of insomnia is characterized by a three point decline in Global PSQI score compared to prior to the treatment.

The Epworth Sleepiness Scale (ESS) is also useful for determining the treatment of insomnia. In scoring the ESS, each item is rated on a 4-point scale from 0=would never doze to 3=high chance of dozing. The item scores are summed to produce a total score (range 0-24). A sum of 10 or more from the 8 individual scores reflects above normal daytime sleepiness and need for further evaluation. In some embodiments, after the treatment the patient experiences a substantial reduction of insomnia that is characterized by at least a one point increase in ESS value compared to prior to the treatment. In some embodiments, the reduction of insomnia is characterized by a one point increase in ESS value compared to prior to the treatment. In some embodiments, the reduction of insomnia is characterized by a two point increase in ESS value compared to prior to the treatment. In some embodiments, the reduction of insomnia is characterized by a three point increase in ESS value compared to prior to the treatment.

The Insomnia Severity Index (ISI) may be used to determine the treatment of insomnia. For example, the Insomnia Severity Index has seven questions, where answers provide a total score ranging from 0 to 28. A total score of 0 to 7 indicates no significant insomnia; 8 to 14 indicates subthreshold insomnia; 15 to 21 indicates clinical insomnia—moderate severity; and 22-28 indicates clinical insomnia—severe. In some embodiments, after the treatment the patient experiences a substantial reduction of insomnia that is characterized by at least a one point decrease in Insomnia Severity Index scale value compared to prior to the treatment. In some embodiments, the reduction of insomnia is characterized by a one point decrease in ISI value compared to prior to the treatment. In some embodiments, the reduction of insomnia is characterized by a two point decrease in ISI value compared to prior to the treatment. In some embodiments, the reduction of insomnia is characterized by a three point decrease in ISI value compared to prior to the treatment.

The Leeds Sleep Evaluation Questionnaire (LSEQ) may be used to determine the treatment of insomnia. The LSEQ is a 10-item, subjective, self-report measure designed to assess changes in sleep quality over the course of a drug treatment intervention. The LSEQ has four domains: Ease of Initiating Sleep (three items), Quality of Sleep (two items), Ease of Waking (two items) and Behavior Following Wakefulness (three items). LSEQ uses a visual analogue scale where the respondents place markers on a group of 10-cm lines representing the changes have experience in a variety of symptoms since beginning treatment. Lines extend between extremes such as “more difficult than usual” and “easier than usual” (item 6 related to ease of waking). Responses are measured using a 100-mm scale and are then averaged to provide a score for each domain. The average scores can be used to evaluate the efficacy and sleep-related side effects of drug treatment. In some embodiments, after the treatment the patient experiences a substantial reduction of insomnia that is characterized by at least about a 10% improvement in total LSEQ value compared to prior to the treatment. In some embodiments, the reduction of insomnia is characterized by an increase in total LSEQ value ranging from about 10% to about 100%, for example, about 10% about, 20%, about 30%, about 40%, about 50%, about 60%, about 70%, about 80%, about 90%, and about 100%, compared to prior to the treatment. In some embodiments, after the treatment the patient experiences a substantial reduction of insomnia that is characterized by at least about a 10% improvement in Ease of Initiating Sleep LSEQ domain value compared to prior to the treatment. In some embodiments, the reduction of insomnia is characterized by an increase in Ease of Initiating Sleep LSEQ domain value ranging from about 10% to about 100%, for example, about 10% about, 20%, about 30%, about 40%, about 50%, about 60%, about 70%, about 80%, about 90%, and about 100%, compared to prior to the treatment. In some embodiments, after the treatment the patient experiences a substantial reduction of insomnia that is characterized by at least about a 10% improvement in Quality of Sleep LSEQ domain value compared to prior to the treatment. In some embodiments, the reduction of insomnia is characterized by an increase in Quality of Sleep LSEQ domain value ranging from about 10% to about 100%, for example, about 10% about, 20%, about 30%, about 40%, about 50%, about 60%, about 70%, about 80%, about 90%, and about 100%, compared to prior to the treatment. In some embodiments, after the treatment the patient experiences a substantial reduction of insomnia that is characterized by at least about a 10% improvement in Ease of Waking LSEQ domain value compared to prior to the treatment. In some embodiments, the reduction of insomnia is characterized by an increase in Ease of Waking LSEQ domain value ranging from about 10% to about 100%, for example, about 10% about, 20%, about 30%, about 40%, about 50%, about 60%, about 70%, about 80%, about 90%, and about 100%, compared to prior to the treatment. In some embodiments, after the treatment the patient experiences a substantial reduction of insomnia that is characterized by at least about a 10% improvement in Behavior Following Wakefulness LSEQ domain value compared to prior to the treatment. In some embodiments, the reduction of insomnia is characterized by an increase in Behavior Following Wakefulness LSEQ domain value ranging from about 10% to about 100%, for example, about 10% about, 20%, about 30%, about 40%, about 50%, about 60%, about 70%, about 80%, about 90%, and about 100%, compared to prior to the treatment.

The Athens Insomnia Scale (AIS) may be used to determine the treatment of insomnia. The AIS scale assess the severity of insomnia using the diagnostic criteria set forth by the International Classification of Diseases (ICD-10). The eight-item questionnaire evaluates sleep onset, night and early-morning waking, sleep time, sleep quality, frequency and duration of complaints, distress cause by the experience and interference with daily functions. Respondents use Likert-type scales to show how severely certain sleep difficulties have affected them in the past month. Scores range from 0 (meaning that the item in question has not been a problem to 3 (indicating more acute sleep difficulties) where answers provide a total score ranging from 0 to 24. In some embodiments, after the treatment the patient experiences a substantial reduction of insomnia that is characterized by at least a one point decrease in total MS value compared to prior to the treatment. In some embodiments, the reduction of insomnia is characterized by a one point decrease in total AIS value compared to prior to the treatment. In some embodiments, the reduction of insomnia is characterized by a two point decrease in total AIS value compared to prior to the treatment. In some embodiments, the reduction of insomnia is characterized by a three point decrease in total AIS value compared to prior to the treatment. In some embodiments, the reduction of insomnia is characterized by a four point decrease in total AIS value compared to prior to the treatment. In some embodiments, the reduction of insomnia is characterized by a five point decrease in total AIS value compared to prior to the treatment. In some embodiments, the reduction of insomnia is characterized by a six point decrease in total AIS value compared to prior to the treatment. In some embodiments, the reduction of insomnia is characterized by a seven point decrease in total AIS value compared to prior to the treatment. In some embodiments, the reduction of insomnia is characterized by an eight point decrease in total AIS value compared to prior to the treatment. In some embodiments, the reduction of insomnia is characterized by a nine point decrease in total AIS value compared to prior to the treatment. In some embodiments, the reduction of insomnia is characterized by a ten point decrease in total AIS value compared to prior to the treatment.

The Sleep Quality Index (SQI) may be used to determine the treatment of insomnia. The SQI is an eight item questionnaire with three categories weighted 0, 1, or 2 for each item (Urponen H. et al. (1991) Sleep Quality and Health: Description of the Sleep Quality Index. In: Peter J. H., Menzel T., Podszus T. von Wichert P. (eds) Sleep and Health Risk. Springer, Berlin, Heidelberg). The value of SQI varies from 0 to 16 with higher scores indicating more severe sleep disturbance. in some embodiments, after the treatment the patient experiences a substantial reduction of insomnia that is characterized by at least a one point decrease in total SQI value compared to prior to the treatment. In some embodiments, the reduction of insomnia is characterized by a one point decrease in total SQI value compared to prior to the treatment. In some embodiments, the reduction of insomnia is characterized by a two point decrease in total SQI value compared to prior to the treatment. In some embodiments, the reduction of insomnia is characterized by a three point decrease in total SQI value compared to prior to the treatment. In some embodiments, the reduction of insomnia is characterized by a four point decrease in total SQI value compared to prior to the treatment. In some embodiments, the reduction of insomnia is characterized by a five point decrease in total SQI value compared to prior to the treatment. In some embodiments, the reduction of insomnia is characterized by a six point decrease in total SQI value compared to prior to the treatment. In some embodiments, the reduction of insomnia is characterized by a seven point decrease in total SQI value compared to prior to the treatment. In some embodiments, the reduction of insomnia is characterized by an eight point decrease in total SQI value compared to prior to the treatment. In some embodiments, the reduction of insomnia is characterized by a nine point decrease in total SQI value compared to prior to the treatment. In some embodiments, the reduction of insomnia is characterized by a ten point decrease in total SQI value compared to prior to the treatment.

Anxious distress has been noted as a prominent feature of both bipolar and major depressive disorder in both primary care and specialty mental health settings. High levels of anxiety have been associated with higher suicide risk, longer duration of illness, and greater likelihood of treatment non-response. In certain embodiments, the dosing frequency and dose amount per administration of Compound 1 are selected to provide therapeutic effects for the treatment of MDD in a patient with anxious distress. In certain embodiments, the dosing frequency and dose amount per administration of Compound 1 are selected to provide therapeutic effects for the treatment of MDD in a patient with mild anxious distress. In certain embodiments, the dosing frequency and dose amount per administration of Compound 1 are selected to provide therapeutic effects for the treatment of MDD in a patient with moderate anxious distress. In certain embodiments, the dosing frequency and dose amount per administration of Compound 1 are selected to provide therapeutic effects for the treatment of MDD in a patient with moderate-severe anxious distress. In certain embodiments, the dosing frequency and dose amount per administration of Compound 1 are selected to provide therapeutic effects for the treatment of MDD in a patient with severe anxious distress.

In some embodiments, the reduction of anxious distress is characterized by an at least one classification reduction in anxious distress severity classification compared to prior to the treatment (e.g., moderate anxious distress to mild anxious distress). In some embodiments, the reduction of anxious distress is characterized by an at least two classification reduction in anxious distress severity classification compared to prior to the treatment. In some embodiments, the reduction of anxious distress is characterized by an at least three classification reduction in anxious distress severity classification compared to prior to the treatment. In some embodiments, the reduction of anxious distress is characterized by a one-classification reduction in anxious distress severity classification compared to prior to the treatment. In some embodiments, the reduction of anxious distress is characterized by a two-classification reduction in anxious distress severity classification compared to prior to the treatment. In some embodiments, the reduction of anxious distress is characterized by a three-classification reduction in anxious distress severity classification compared to prior to the treatment. In some embodiments, the reduction of anxious distress is characterized by a four-classification reduction in anxious distress severity classification compared to prior to the treatment.

In some embodiments, the dosing frequency and dose amount per administration of Compound 1 are selected to provide therapeutic effects for the treatment of depression and that do not substantially sedate the patient (i.e., the MDD is treated without substantially sedating the treated patient). In certain embodiments, the dosing frequency and dose amount per administration of Compound 1 are selected to provide therapeutic effects for the treatment of MDD and that do not substantially sedate the patient. In certain embodiments, the dosing frequency and dose amount per administration of Compound 1 are selected to provide therapeutic effects for the treatment of moderate MDD and that do not substantially sedate the patient. In certain embodiments, the dosing frequency and dose amount per administration of Compound 1 are selected to provide therapeutic effects for the treatment of severe MDD and that do not substantially sedate the patient.

A patient's sedation level may be measured using methods that are known to those skilled in the art. For example, sedation level may be measured using the Modified Observer's Assessment of Alertness/Sedation Scale (G. Schmidt, et al., Comparative Evaluation of the Datex-Ohmeda S/5 Entropy Module and the Bispectral Index® Monitor during Propofol-Remifentanil Anesthesia. Anesthesiology 2004; 101:1283-90) or the Stanford Sleepiness Scale (Quantification of Sleepiness: A New Approach. Psychophysiology Volume 10, Issue 4, pages 431-436, July 1973).

In certain embodiments, the dosing frequency and dose amount per administration of Compound 1 are selected to provide therapeutic effects for the treatment of depression and a Modified Observer's Assessment of Alertness/Sedation Scale (MOAS/S) score of at least 4.0. In certain embodiments, the dosing frequency and dose amount per administration of Compound 1 are selected to provide therapeutic effects for the treatment of depression and a MOAS/S score of 4. In certain embodiments, the dosing frequency and dose amount per administration of Compound 1 are selected to provide therapeutic effects for the treatment of depression and a MOAS/S score of 5.

In certain embodiments, the dosing frequency and dose amount per administration of Compound 1 are selected to provide therapeutic effects for the treatment of depression and a Stanford Sleepiness Scale Score of less than 3.0. In certain embodiments, the dosing frequency and dose amount per administration of Compound 1 are selected to provide therapeutic effects for the treatment of depression and a Stanford Sleepiness Scale Score of 2. In certain embodiments, the dosing frequency and dose amount per administration of Compound 1 are selected to provide therapeutic effects for the treatment of depression and a Stanford Sleepiness Scale Score of 1.

According to some embodiments of the present invention, the dosing frequency and dose amount per administration of Compound 1 are selected to provide therapeutic effects for the treatment of a mood or affective disorder selected from perimenopause, generalized anxiety disorder, panic disorder, social anxiety disorder, acute stress disorder, post-traumatic stress disorder, specific phobia, and selective mutism.

In some embodiments, the dosing frequency and dose amount per administration of Compound 1 are selected to provide therapeutic effects for the treatment of perimenopause. In some embodiments, the dosing frequency and dose amount per administration of Compound 1 are selected to provide therapeutic effects for the treatment of perimenopausal depression. Methods of diagnosing perimenopausal depression are known to those skilled in the art, such as set forth in Pauline M. Maki, et al. Guidelines for the Evaluation and Treatment of Perimenopausal Depression: Summary and Recommendations. Journal of Women's Health (DOI: 10.1089/jwh.2018.27099.mensocrec). In some embodiments, the dosing frequency and dose amount per administration of Compound 1 are selected to provide therapeutic effects for the treatment of perimenopause anxiety. In some embodiments, the dosing frequency and dose amount per administration of Compound 1 are selected to provide therapeutic effects for the treatment of perimenopause agitation.

In some embodiments, the dosing frequency and dose amount per administration of Compound 1 are selected to provide therapeutic effects for the treatment of menopause anxiety or postmenopause anxiety.

In some embodiments, the dosing frequency and dose amount per administration of Compound 1 are selected to provide therapeutic effects for the treatment of menopause agitation or postmenopause agitation.

In some embodiments, the dosing frequency and dose amount per administration of Compound 1 are selected to provide therapeutic effects for the acute treatment of depression. In some embodiments, the dosing frequency and dose amount per administration of Compound 1 are selected to provide therapeutic effects for the acute treatment of depression and, after the acute treatment of depression, Compound 1 is no longer administered and a dosing frequency and dose amount of second anti-depressant agent is selected to provide therapeutic effects for the chronic treatment of depression.

In some embodiments, the dosing frequency and dose amount per administration of Compound 1 are selected to provide therapeutic effects for the acute treatment of depression and, after the acute treatment of depression, a dosing frequency and dose amount of Compound 1 is selected to provide therapeutic effects for the chronic treatment of depression. In certain embodiments, the daily dosing of Compound 1 for the acute treatment of depression is greater than the daily dosing of Compound 1 for the chronic treatment of depression.

In some embodiments, the dosing frequency and dose amount per administration of Compound 1 are selected to prevent the recurrence of depression. In some embodiments, the dosing frequency and dose amount per administration of Compound 1 are selected to maintain remission of depression.

In certain embodiments, Compound 1 or a pharmaceutically acceptable salt thereof is administered on a once a day or twice a day basis for at least a week, for example, about a week, about 2 weeks, about 3 weeks, about 4 weeks, about 5 weeks, about 6 weeks, about 7 weeks, about 8 weeks, about 9 weeks, about 10 weeks, about 12 weeks, about 18 weeks, about 24 weeks, and about 50 weeks.

In certain embodiments, at least about 5 mg or about 5 mg of Compound 1 or a pharmaceutically acceptable salt thereof is administered on a once a day or twice a day basis for at least a week. In certain embodiments, at least about 10 mg or about 10 mg of Compound 1 or a pharmaceutically acceptable salt thereof is administered on a once a day or twice a day basis for at least a week. In certain embodiments, at least about 15 mg or about 15 mg of Compound 1 or a pharmaceutically acceptable salt thereof is administered on a once a day or twice a day basis for at least a week. In certain embodiments, at least about 20 mg or about 20 mg of Compound 1 or a pharmaceutically acceptable salt thereof is administered on a once a day or twice a day basis for at least a week. In certain embodiments, at least about 25 mg or about 25 mg of Compound 1 or a pharmaceutically acceptable salt thereof is administered on a once a day or twice a day basis for at least a week. In certain embodiments, at least about 30 mg or about 30 mg of Compound 1 or a pharmaceutically acceptable salt thereof is administered on a once a day or twice a day basis for at least a week. In certain embodiments, at least about 35 mg or about 35 mg of Compound 1 or a pharmaceutically acceptable salt thereof is administered on a once a day or twice a day basis for at least a week. In certain embodiments, at least about 40 mg or about 40 mg of Compound 1 or a pharmaceutically acceptable salt thereof is administered on a once a day or twice a day basis for at least a week. In certain embodiments, at least about 45 mg or about 45 mg of Compound 1 or a pharmaceutically acceptable salt thereof is administered on a once a day or twice a day basis for at least a week. In certain embodiments, at least about 50 mg or about 50 mg of Compound 1 or a pharmaceutically acceptable salt thereof is administered on a once a day or twice a day basis for at least a week. In certain embodiments, at least about 55 mg or about 55 mg of Compound 1 or a pharmaceutically acceptable salt thereof is administered on a once a day or twice a day basis for at least a week. In certain embodiments, at least about 60 mg or about 60 mg of Compound 1 or a pharmaceutically acceptable salt thereof is administered on a once a day or twice a day basis for at least a week. In certain embodiments, at least about 65 mg or about 65 mg of Compound 1 or a pharmaceutically acceptable salt thereof is administered on a once a day basis for at least a week. In certain embodiments, at least about 70 mg or about 70 mg of Compound 1 or a pharmaceutically acceptable salt thereof is administered on a once a day basis for at least a week. In certain embodiments, at least about 75 mg or about 75 mg of Compound 1 or a pharmaceutically acceptable salt thereof is administered on a once a day basis for at least a week. In certain embodiments, at least about 80 mg or about 80 mg of Compound 1 or a pharmaceutically acceptable salt thereof is administered on a once a day basis for at least a week. In certain embodiments, at least about 85 mg or about 85 mg of Compound 1 or a pharmaceutically acceptable salt thereof is administered on a once a day basis for at least a week. In certain embodiments, at least about 90 mg or about 90 mg of Compound 1 or a pharmaceutically acceptable salt thereof is administered on a once a day basis for at least a week. In certain embodiments, at least about 95 mg or about 95 mg of Compound 1 or a pharmaceutically acceptable salt thereof is administered on a once a day basis for at least a week. In certain embodiments, at least about 100 mg or about 100 mg of Compound 1 or a pharmaceutically acceptable salt thereof is administered on a once a day basis for at least a week. In certain embodiments, at least about 105 mg or about 105 mg of Compound 1 or a pharmaceutically acceptable salt thereof is administered on a once a day basis for at least a week. In certain embodiments, at least about 110 mg or about 0.110 mg of Compound 1 or a pharmaceutically acceptable salt thereof is administered on a once a day basis for at least a week. In certain embodiments, at least about 115 mg or about 115 mg of Compound 1 or a pharmaceutically acceptable salt thereof is administered on a once a day basis for at least a week. In certain embodiments, at least about 120 mg or about 120 mg of Compound 1 or a pharmaceutically acceptable salt thereof is administered on a once a day basis for at least a week.

According to some embodiments, the substantial reduction in depression provided by the methods of the present disclosure requires treatment for a specified time interval (e.g., at least one week) before the patient experiences substantial reduction of depression (i.e., there is an induction period before the patient experiences a substantial reduction in depression). In some embodiments, after treatment for at least one week, at least two weeks, at least three weeks, at least four weeks, at least five weeks, at least six weeks, at least seven weeks or at least eight weeks, the patient experiences a substantial reduction of depression compared to prior to the treatment. In certain embodiments, after treatment for at least one week the patient experiences a substantial reduction of depression compared to prior to the treatment. According to this embodiment, the substantial reduction in depression may be expressed using any of the methods described herein (for example, decline in total Hamilton Depression Rating Scale (HAM-D) value compared to prior to the treatment, improvement in the Montgomery Asberg Depression Rating Scale value compared to prior to the treatment, etc.).

The HAM-D is a depression rating scale consisting of 17 items, eight items are scored on a 5-point scale (ranging from 0 to 4), and 9 items are scores on a 3-point scale (ranging from 0 to 2). The total score of the 17 items ranges from 0 to 50 with higher scores indicating greater depression. The total score the 17 items is used to categorize the severity of depression: normal (total score between 0 and 7), mild depression (total score between 8 and 13), moderate depression (total score between 14-18), severe depression (total score between 19-22). Therefore, a decrease in the total score or on individual item scores indicates improvement Hamilton, M. A Rating Scale for Depression, Journal of Neurology, Neurosurgery, and Psychiatry. (1960) 23, pages 56-62.

In some embodiments, after the treatment the patient experiences a substantial reduction of depression that is characterized by at least about a 30% decline in total Hamilton Depression Rating Scale (HAM-D) value compared to prior to the treatment. In some embodiments, the reduction of depression is characterized by a decline in HAM-D value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of depression that is characterized by at least a one point decline in HAM-D value compared to prior to the treatment. In some embodiments, the reduction of depression is characterized by a decline in HAM-D value ranging from about one point to about twenty points, for example, about one point, about two points, about three points, about four points, about five points, about six points, about seven points, about eight points, about nine points, about ten points, about eleven points, about twelve points, about thirteen points, about fourteen points, about fifteen points, about sixteen points, about seventeen points, about eighteen points, about nineteen points, and about twenty points compared to prior to the treatment. In some embodiments, the reduction of depression is characterized by a decline HAM-D value of about two points. In some embodiments, the reduction of depression is characterized by a decline in HAM-D value of about three points. In some embodiments, the reduction of depression is characterized by a decline in HAM-D value of about four points. In some embodiments, the reduction of depression is characterized by a decline in HAM-D value of about five points. In some embodiments, the reduction of depression is characterized by a decline HAM-D value of about six points. In some embodiments, the reduction of depression is characterized by a decline in HAM-D value of about seven points. In some embodiments, the reduction of depression is characterized by a decline in HAM-D value of about eight points. In some embodiments, the reduction of depression is characterized by a decline in HAM-D value of about nine points. In some embodiments, the reduction of depression is characterized by a decline HAM-D value of about ten points. In some embodiments, the reduction of depression is characterized by a decline in HAM-D value of about eleven points. In some embodiments, the reduction of depression is characterized by a decline in HAM-D value of about twelve points. In some embodiments, the reduction of depression is characterized by a decline in HAM-D value of about thirteen points. In some embodiments, the reduction of depression is characterized by a decline HAM-D value of about fourteen points. In some embodiments, the reduction of depression is characterized by a decline in HAM-D value of about fifteen points. In some embodiments, the reduction of depression is characterized by a decline in HAM-D value of about sixteen points. In some embodiments, the reduction of depression is characterized by a decline in HAM-D value of about seventeen points. In some embodiments, the reduction of depression is characterized by a decline HAM-D value of about eighteen points. In some embodiments, the reduction of depression is characterized by a decline in HAM-D value of about nineteen points. In some embodiments, the reduction of depression is characterized by a decline in HAM-D value of about twenty points.

In some embodiments, after the treatment the patient experiences a substantial reduction of depression that is characterized by at least a one category change in HAM-D severity classification compared to prior to the treatment. In some embodiments, the reduction of depression is characterized by a one category change HAM-D severity classification compared to prior to the treatment. In some embodiments, the reduction of depression is characterized by a two category change HAM-D severity classification compared to prior to the treatment. In some embodiments, the reduction of depression is characterized by a three category change HAM-D severity classification compared to prior to the treatment. In certain embodiments, the reduction of depression is characterized by remission according to HAM-D value after said treatment (i.e., total HAM-D value of 7 or less).

The Montgomery Asberg Depression Rating Scale (MADRS) is a depression rating scale consisting of 10 items, each rated 0 to 6. The 10 items represent the core symptoms of depressive illness. The total score of the 10 items ranges from 0 to 60. Decrease in the total score or on individual items indicates improvement (Montgomery S. A. and Åsberg M. A, New Depression Scale Designed to be Sensitive to Change, Br. J. Psychiatry. (1979) April; 134, pages 382-9.).

In some embodiments, after the treatment the patient experiences a substantial reduction of depression that is characterized by at least about a 30% decline in Montgomery Asberg Depression Rating Scale (MADRS) compared to prior to the treatment. In some embodiments, the reduction of depression is characterized by a decline in MADRS value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of depression that is characterized by at least a one point decline in MADRS value compared to prior to the treatment. In some embodiments, the reduction of depression is characterized by a decline in MADRS value ranging from about one point to about five points, for example, about one point, about two points, about three points, about four points, and about five points compared to prior to the treatment. In some embodiments, the reduction of depression is characterized by a decline MADRS value of about two points. In some embodiments, the reduction of depression is characterized by a decline in MADRS value of about three points. In some embodiments, the reduction of depression is characterized by a decline in MADRS value of about four points. In some embodiments, the reduction of depression is characterized by a decline in MADRS value of about five points. In certain embodiments, the reduction of depression is characterized by remission according to MADRS value after said treatment (i.e., MADRS value of 12 or less).

The Hamilton Rating Scale for Anxiety (HAM-A) is an anxiety rating scale consisting of 14 items that assess anxious mood, tension, fear, insomnia, intellectual (cognitive) functioning, depressed mood, behavior at interview, somatic (sensory), cardiovascular, respiratory, gastrointestinal, genitourinary, autonomic, and somatic (muscular) symptom (Hamilton, M. The Assessment of Anxiety States by Rating, Br J Med Psychol. (1959); 32 (1), pages 50-5). Each symptom is rated from 0 (absent) to 4 (maximum severity) scale. The total score is used to categorize the severity of anxiety: mild severity (total score less than 17), mild to moderate severity (total score between 18-24), and moderate to severe (total score between 25-30). Total scores range from 0 to 56 with higher scores indicating greater severity.

In some embodiments, after the treatment the patient experiences a substantial reduction of depression that is characterized by at least about a 30% decline in total HAM-A value compared to prior to the treatment. In some embodiments, the reduction of anxiety is characterized by a decline in HAM-A value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of anxiety that is characterized by at least a one point decline in HAM-A value compared to prior to the treatment. In some embodiments, the reduction of anxiety is characterized by a decline in HAM-A value ranging from about one point to about five points, for example, about one point, about two points, about three points, about four points, and about five points compared to prior to the treatment. In some embodiments, the reduction of anxiety is characterized by a decline HAM-A value of about two points. In some embodiments, the reduction of anxiety is characterized by a decline in HAM-A value of about three points. In some embodiments, the reduction of anxiety is characterized by a decline in HAM-A value of about four points. In some embodiments, the reduction of depression is characterized by a decline in HAM-A value of about five points.

In some embodiments, after the treatment the patient experiences a substantial reduction of anxiety that is characterized by at least a one category change in HAM-A severity classification compared to prior to the treatment. In some embodiments, the reduction of anxiety is characterized by a one category change HAM-A severity classification compared to prior to the treatment. In some embodiments, the reduction of anxiety is characterized by a two category change HAM-A severity classification compared to prior to the treatment. In some embodiments, the reduction of depression is characterized by a three category change HAM-A severity classification compared to prior to the treatment.

In some embodiments, after the treatment the patient experiences a substantial reduction of depression that is characterized by partial remission of the patient's depression. In some embodiments, after the treatment the patient experiences a substantial reduction of MDD that is characterized by partial remission of the patient's depression. In certain embodiments, partial remission of MDD is where the symptoms of the immediately previous major depressive episode are present, but full criteria are not met, or there is a period lasting less than 2 months without any significant symptoms of a major depressive episode following the end of such an episode (i.e., the DSM-5's definition of partial remission).

In some embodiments, after the treatment the patient experiences a substantial reduction of depression that is characterized by full remission of the patient's depression. In some embodiments, after the treatment the patient experiences a substantial reduction of MDD that is characterized by full remission of the patient's depression. In certain embodiments, full remission is where during the past 2 months, no significant signs or symptoms of the disturbance were present (i.e., the DSM-5's definition of full remission).

The Clinical Global Impression (CGI) (Guy 1976 (Guy W (1976), ECDEU Assessment Manual for Psychopharmacology, Revised. Rockville, Md.: US Department of Health, Education and Welfare) consists of three subscales: the CGI-Severity (CGI-S), the CGI-Improvement (CGI-I) and Efficacy Index. The CGI-S assesses the clinician's impression of the patient's current mental illness. A treating clinician categorizes the severity of the patient's current mental illness on a 7-point scale: 1 (normal, not at all ill), 2 (borderline mentally ill), 3 (mildly ill), 4 (moderately ill), 5 (markedly ill), 6 (severely ill), and 7 (among the most extremely ill patients). The CGI-I assesses the participant's improvement (or worsening) from baseline. A treating clinician categorizes the patient's condition relative to Baseline (e.g., prior to administering an antidepressant) on a 7-point scale: 1 (very much improved), 2 (much improved), 3 (minimally improved), 4 (no change), 5 (minimally worse), 6 (much worse), and 7 (very much worse).

In some embodiments, after the treatment the patient experiences a substantial reduction of depression that is characterized by at least a one point decline in CGI-S value compared to prior to the treatment. In some embodiments, the reduction of depression is characterized by a one point decline in CGI-S value compared to prior to the treatment. In some embodiments, the reduction of depression is characterized by a two point decline in CGI-S value compared to prior to the treatment. In some embodiments, the reduction of depression is characterized by a three point decline in CGI-S value compared to prior to the treatment.

In some embodiments, after the treatment the patient experiences a substantial reduction of depression that is characterized by at least a one point decline in CGI-I value compared to prior to the treatment. In some embodiments, the reduction of depression is characterized by a one point decline in CGI-I value compared to prior to the treatment. In some embodiments, the reduction of depression is characterized by a two point decline in CGI-I value compared to prior to the treatment. In some embodiments, the reduction of depression is characterized by a three point decline in CGI-I value compared to prior to the treatment.

The Symptoms of Depression Questionnaire (SDQ) is a 44-item, self-report scale that consists of five subscales: SDQ-1, SDQ-2, SDQ-3, SDQ-4 and SDQ-5. SDQ-1 includes items related to lassitude, mood, and cognitive functioning. SDQ-2 includes items related to anxiety, agitation, irritability, and anger. SDQ-3 includes items related to suicidal ideation. SDQ-4 assesses disruptions in sleep quality. SDQ-5 includes items on changes in appetite and weight. SDQ is used to assess symptom severity across several subtypes of depression (Pedrelli, P., et al., Reliability and Validity of the Symptoms of Depression Questionnaire (SDQ), CNS Spectr. 2014 December; 19(6), pages 535-546.). The items are rated on a 6-point scale. Each item is rated based on a participant's perception of what is normal for the individual (score=2), what is better than normal (score=1), and what is worse than normal (scores=3-6). Total scores range from 0 to 264 with higher scores indicating greater severity.

In some embodiments, after the treatment the patient experiences a substantial reduction of depression that is characterized by at least about a 10% decline in total SDQ scale value compared to prior to the treatment. In some embodiments, the reduction of depression is characterized by a decline in total SDQ scale value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of depression that is characterized by at least about a 10% decline in at least one subscale value selected from SDQ-1, SDQ-2, SDQ-3, SDQ-4 and SDQ-5 compared to prior to the treatment. In some embodiments, the reduction of depression is characterized by a decline in at least one subscale value selected from SDQ-1, SDQ-2, SDQ-3, SDQ-4 and SDQ-5 value ranging from about 10% to about 100%, for example, about 20%, about 30%, about 40%, about 50%, about 60%, about 70%, about 80%, about 90%, and about 100%, compared to prior to the treatment.

In some embodiments, after the treatment the patient experiences a substantial reduction of depression that is characterized by at least a one point decline in Global PSQI (described above) score compared to prior to the treatment. In some embodiments, the reduction of depression is characterized by a one point decline in Global PSQI score compared to prior to the treatment. In some embodiments, the reduction of depression is characterized by a two point decline in Global PSQI score compared to prior to the treatment. In some embodiments, the reduction of depression is characterized by a three point decline in Global PSQI score compared to prior to the treatment.

In some embodiments, the method of treating depression further includes a step of titrating the dose of Compound 1 until a maintenance dose is achieved in the patient. In some embodiments, the titration is conducted for at least about one week until a maintenance dose is achieved in the patient. In one embodiment, the titration is conducted for about 2 weeks until a maintenance dose is achieved in the patient. In some embodiments, the titration is conducted for about 7 days to about 30 days until a maintenance dose is achieved in the patient. In some embodiments, the titration is conducted for about 12 days to about 20 days until a maintenance dose is achieved in the patient. In some embodiments, during the titration step, a constant daily dose of Compound 1 is provided. In further embodiments, the constant daily dose of Compound 1 is provided for at least two weeks.

The daily dose can be titrated in one or more steps. The daily dosage can be titrated by increasing a single daily dosage, or each dose of a twice-daily dosing regimen. The amount a dosage is stepped, where there are multiple titration steps, can be the same, or can be different.

In some embodiments, the titration is initiated with from about 15 mg to about 100 mg of Compound 1 or a pharmaceutically acceptable salt thereof, including about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg and about 100 mg including all ranges there between once or twice daily. In some embodiments, the titration is initiated with about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg and about 100 mg of Compound 1 or a pharmaceutically acceptable salt thereof once or twice daily. In certain embodiments, doses can be adjusted in 5-30 mg increments every 1 to 4 days. In certain embodiments, doses can be adjusted in 5-30 mg increments every week. In some embodiments, the titration is conducted for at least about one week, 2 weeks, 3 weeks, 4 weeks, 5 weeks, 6 weeks, about 7 weeks, about 8 weeks, about 9 weeks or about 10 weeks prior to the maintenance dose

In certain embodiments, ascending doses of Compound 1 are administered during the titration until a maintenance dose is achieved in the patient. In certain embodiments, ascending doses of the Compound 1 are administered during the titration until an effective amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg is achieved in the patient.

In certain embodiments, patients are initially administered about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg or about 100 mg of Compound 1 or a pharmaceutically acceptable salt once or twice a day and are titrated to a maintenance dose of about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg once or twice a day. In certain embodiments, patients are initially administered from about 15 mg to about 100 mg of Compound 1 or a pharmaceutically acceptable salt, including about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg and about 100 mg, including all ranges there between once or twice a day and are titrated to a maintenance dose of from about 20 mg to about 120 mg, including about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg including all ranges therebetween, once or twice a day.

In some embodiments, the present disclosure provides a method of treating depression that includes the steps of: (a) administering an initial daily dose of Compound 1 for at least one week and (b) administering a maintenance daily dose for at least one week. In certain embodiments, the initial daily dose is greater than the maintenance daily dose. In certain embodiments, the initial daily dose is less than the maintenance daily dose. In certain embodiments, the initial daily dose is administered for two weeks and the maintenance daily dose is administered is administered for at least one month.

In some embodiments, the present disclosure provides a method of treating depression that includes the steps of:

(a) administering a loading dose of Compound 1 and

(b) administering a maintenance dose of Compound 1.

In some embodiments, the loading dose is administered for about 1 day to about 14 days, including about 1 day, about 2 days, about 3 days, about 4 days, about 5 days, about 6 days, about 7 days, about 8 days, about 9 days, about 10 days, about 11 days, about 12 days, about 13 days and about 14 days, including all ranges therebetween. In some embodiments, the loading dose is administered for about 1 day, about 2 days, about 3 days, about 4 days, about 5 days, about 6 days, about 7 days, about 8 days, about 9 days, about 10 days, about 11 days, about 12 days, about 13 days or about 14 days.

In some embodiments, the methods comprise a loading dose of from about 30 mg to about 120 mg, including about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg including all ranges therebetween. In some embodiments, the methods comprise a loading dose of about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg.

In some embodiments, the maintenance dose is administered for from about 1 month to about 36 months, including about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, or about 36 months including all ranges there between. In some embodiments, the maintenance dose is administered for about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, or about 36 months.

In some embodiments, the methods comprise a maintenance dose of from about 30 mg to about 120 mg, including about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, and about 120 mg including all ranges therebetween, once or twice a day. In some embodiments, the methods comprise a maintenance dose of from about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg, once or twice a day.

In some embodiments, the loading dose administration methods further comprise a cessation period after administration of the loading dose and prior to administration of the maintenance dose.

In some embodiments, the cessation period is about one day, about two days, about three days, about four days, about five days, about six days, or about seven days. In some embodiments, the cessation period is from about one day to about seven days, including about one day, about two days, about three days, about four days, about five days, about six days, and about seven days, including all ranges there between.

In some embodiments, the cessation period is about one week, about two weeks, about three weeks, about four weeks, about five weeks, about six weeks, about seven weeks, or about eight weeks. In some embodiments, the cessation period is from about one week to about eight weeks, including about one week, about two weeks, about three weeks, about four weeks, about five weeks, about six weeks, about seven weeks, and about eight weeks including all ranges there between.

In some embodiments, the methods of the present disclosure comprise continuous administration of Compound 1 or a pharmaceutically acceptable salt thereof for a specified interval (for example, one week) followed by a cessation period wherein the patient is not administered Compound 1. In some embodiments, the methods of the present disclosure comprise continuous administration of Compound 1 or a pharmaceutically acceptable salt thereof for from about one week to about eight weeks, including about one week, about two weeks, about three weeks, about four weeks, about five weeks, about six weeks, about seven weeks, and about eight weeks including all ranges there between. In some embodiments, the methods of the present disclosure comprise continuous administration of Compound 1 or a pharmaceutically acceptable salt thereof for about one week, about two weeks, about three weeks, about four weeks, about five weeks, about six weeks, about seven weeks, or about eight weeks.

In some embodiments, the methods of the present disclosure comprise continuous administration of Compound 1 or a pharmaceutically acceptable salt thereof for from about one month to about 36 months, including about one month, about two months, about three months, about four months, about five months, about six months, about seven months, about eight months, about 9 months, about 10 months, about 11 months, about 12 months, about 18 months, about 24 months, about 30 months, and about 36 months including all ranges there between. In some embodiments, the methods of the present disclosure comprise continuous administration of Compound 1 or a pharmaceutically acceptable salt thereof for about one month, about two months, about three months, about four months, about five months, about six months, about seven months, about eight months, about nine months, about ten months, about eleven months, about twelve months, about 18 months, about 24 months, about 30 months, or about 36 months.

In some embodiments, the methods of the present disclosure comprise intermittent administration of Compound 1 or a pharmaceutically acceptable salt thereof. As used herein, intermittent administration means cycling a patient in need thereof on and off treatment with Compound 1 or a pharmaceutically acceptable salt thereof for specified time intervals.

In some embodiments, intermittent administration comprises:
(a) administering Compound 1 for a first administration period;
(b) after the first administration period (a), not administering Compound 1 for a cessation period;
(c) after the cessation period (b), administering Compound 1 for a second administration period.

In some embodiments, the intermittent administration further comprises one or more additional cessation periods (for example, a second cessation period) and/or administration periods (for example, a third administration period). In such embodiments, the present disclosure contemplates embodiments wherein the additional cessation and/or administration periods have the durations described herein for the first administration period and the cessation period.

In some embodiments, two or more of the periods (a), (b) and (c) are the same (for example, the first administration period, cessation period and second administration period are each one week). In some embodiments, the periods (a) and (b) (for example, one week) are the same and the period (c) is different (for example, two weeks). In some embodiments, the periods (a) and (c) are the same and the period (b) is different. In some embodiments, the periods (b) and (c) are the same and the period (a) is different. In some embodiments, the periods (a), (b) and (c) are the different (for example, the first administration period is one week, the cessation period is two weeks and the second administration period is three weeks).

In some embodiments, the first administration period is about one week, about two weeks, about three weeks, about four weeks, about five weeks, about six weeks, about seven weeks, or about eight weeks. In some embodiments, the first administration period is from about one week to about eight weeks, including about one week, about two weeks, about three weeks, about four weeks, about five weeks, about six weeks, about seven weeks, and about eight weeks including all ranges there between.

In some embodiments, the cessation period is about one week, about two weeks, about three weeks, about four weeks, about five weeks, about six weeks, about seven weeks, or about eight weeks. In some embodiments, the cessation period is from about one week to about eight weeks, including about one week, about two weeks, about three weeks, about four weeks, about five weeks, about six weeks, about seven weeks, and about eight weeks including all ranges there between.

In some embodiments, the second administration period is about one week, about two weeks, about three weeks, about four weeks, about five weeks, about six weeks, about seven weeks, or about eight weeks. In some embodiments, the second administration period is from about one week to about eight weeks, including about one week, about two weeks, about three weeks, about four weeks, about five weeks, about six weeks, about seven weeks, or about eight weeks including all ranges there between.

In some embodiments, the first administration period, cessation period and second administration period are one week. In some embodiments, the first administration period, cessation period and second administration period are two weeks. In some embodiments, the first administration period, cessation period and second administration period are three weeks. In some embodiments, the first administration period, cessation period and second administration period are four weeks. In some embodiments, the first administration period, cessation period and second administration period are five weeks. In some embodiments, the first administration period, cessation period and second administration period are six weeks. In some embodiments, the first administration period, cessation period and second administration period are seven weeks. In some embodiments, the first administration period, cessation period and second administration period are eight weeks.

In some embodiments, the first administration period is about one week; the cessation period is about three weeks; and the second administration period is about one week.

In some embodiments, the first administration period is about two weeks; the first cessation period is about two weeks; the second administration period is about one week; the second cessation period is about one week and the third administration period is about one week.

In some embodiments, the intermittent administration period (including the administration and cessation periods) is about one month, about two months, about three months, about four months, about five months, about six months, about seven months, about eight months, about nine months, about ten months, about eleven months, about twelve months, about 18 months, about 24 months, about 30 months or about 36 months. In some embodiments, the intermittent administration period is from about one month to about twelve months, including about two months, about three months, about four months, about five months, about six months, about seven months, about eight months, about nine months, about ten months, about eleven months, about twelve months, about 18 months, about 24 months, about 30 months and about 36 months, including all ranges there between.

In some embodiments of the present disclosure, Compound 1 or a pharmaceutically acceptable salt thereof is administered to a patient with food. In some embodiments, Compound 1 or a pharmaceutically acceptable salt thereof is administered to a patient about 5 minutes, about 10 minutes, about 15 minutes, about 30 minutes, about 45 minutes, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours or about 8 hours after food is ingested.

In some embodiments, the food ingested is a high fat and high calorie food. In some embodiments, the caloric content of the high fat and high calorie food is at least about 700 kilocalories (kcal), and at least about 40 percent of the caloric content of the food is from fat. For example, the fat can contribute to about 50 percent of the caloric content of the food of high fat and high calorie. In some embodiments, the caloric content of the high fat and high calorie food is about 900 kilocalories.

In some embodiments, the food ingested is a medium fat and medium calorie food. In some embodiments, the caloric content of the medium fat and medium calorie food is about 300 kcal to about 700 kcal, and between about 20 percent to about 40 percent of the caloric content of the food is from fat. In some embodiments, the caloric content of the medium fat and medium calorie food is about 400 kcal.

In some embodiments, the food ingested is a low fat and low calorie food. In some embodiments, the caloric content of the low fat and low calorie food is between about 100 kcal to about 300 kcal, and the fat content is approximately 3 grams or less, or about 20 percent or less of the caloric content of the food are from fat. In some embodiments, the caloric content of the food of low fat and low calorie is about 100 kilocalories.

In some embodiments of the present disclosure, Compound 1 or a pharmaceutically acceptable salt thereof is administered to a patient after a fasting period. In some embodiments, Compound 1 or a pharmaceutically acceptable salt thereof is administered to a patient after a fasting period of about 1 hour, about 2 hours, about 3 hours, about 4 hours, about 5 hours, about 6 hours, about 7 hours, about 8 hours, about 9 hours, about 10 hours, about 11 hours, or about 12 hours.

In some embodiments of the present disclosure, Compound 1 or a pharmaceutically acceptable salt thereof is administered to a patient without regard to meals. In some embodiments, Compound 1 or a pharmaceutically acceptable salt thereof is administered to a patient at bedtime.

In some embodiments, the methods of the present disclosure comprise controlling the gastrointestinal pH of the patient prior to, concurrently with or after administration of Compound 1. In some embodiments, the gastrointestinal pH of the patient is controlled prior to administration of Compound 1. In some embodiments, the gastrointestinal pH of the patient is controlled after administration of Compound 1.

In some embodiments, the pH is controlled by administering a drug, food or liquid to a patient that decreases gastrointestinal pH prior to, concurrently with or after administration of Compound 1. In some embodiments, the liquid is an acidic beverage (such as a carbonated beverage).

In some embodiments, the pH is controlled by the patient avoiding a drug, food or beverage that increases gastrointestinal pH prior to, concurrently with or after administration of Compound 1. In some embodiments, the drug that increases gastrointestinal pH is a proton pump inhibitor or an orally-administered antacid.

In some embodiments, the initial daily dosing frequency and dose amount per administration of Compound 1 are selected to provide therapeutic effects for the acute treatment of depression and the maintenance daily dosing frequency and dose amount per administration of Compound 1 are selected to provide therapeutic effects for the chronic treatment of depression.

In some embodiments, the initial daily dosing frequency and dose amount per administration of Compound 1 are selected to provide therapeutic effects for the acute treatment of depression and the maintenance daily dosing frequency and dose amount per administration of Compound 1 are selected to maintain remission of depression.

In some embodiments, the initial daily dosing frequency and dose amount per administration of Compound 1 are selected to provide therapeutic effects for the acute treatment of depression and the maintenance the daily dosing frequency and dose amount per administration of Compound 1 are selected to prevent recurrence of depression.

In certain embodiments, the initial daily dose is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg or about 120 mg and the maintenance daily dose is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, or about 115 mg provided the initial daily dose is greater than the maintenance daily dose.

According to some embodiments of the present invention, the methods of the present invention provide therapeutically effective blood plasma levels of Compound 1 for treating depression. Blood plasma levels of Compound 1 may be expressed using pharmacokinetic parameters that are known to those skilled in the art, such as steady state plasma levels, AUC, Cmax and Cmin. Throughout the present disclosure pharmacokinetic parameters are described in terms of providing a steady state plasma level of a particular PK parameter (such as steady state plasma Cmax, steady state AUC, etc.). However, the present disclosure contemplates embodiments where the steady state PK parameters that are expressed herein are average values from a patient population (such as a mean value). Thus, the following description of pharmacokinetic parameters describes mean steady state PK parameter values as well values from an individual patient.

In some embodiments, the present methods provide steady state plasma levels of Compound 1 that correlate to one or more statistically significant therapeutic effects. In certain embodiments, the therapeutically effective steady state plasma levels of Compound 1 provided by the methods of the present invention range from about 1 ng/mL to about 200 ng/mL, including about 1 ng/ml, about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL about 80 ng/mL, about 85 ng/mL, about 90 ng/mL, about 95 ng/mL, about 100 ng/mL, about 105 ng/mL, about 110 ng/mL, about 115 ng/mL, about 120 ng/mL, about 125 ng/mL, about 130 ng/mL, about 135 ng/mL, about 140 ng/mL, about 145 ng/mL, about 150 ng/mL, about 155 ng/mL, about 160 ng/mL, about 165 ng/mL, about 170 ng/mL, about 175 ng/mL about 180 ng/mL, about 185 ng/mL, about 190 ng/mL, about 195 ng/mL, and 200 ng/ml, including all ranges there between. In certain embodiments, the therapeutically effective steady state plasma levels of Compound 1 provided by the methods of the present invention range from about 50 ng/ml to 200 ng/ml.

In certain embodiments, the therapeutically effective steady state plasma levels of Compound 1 is provided by administering a daily dose of Compound 1 or a pharmaceutically acceptable salt thereof of about 15 mg. In further embodiments, the therapeutically effective steady state plasma levels of Compound 1 is provided by administering about 15 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 20 mg. In further embodiments, the therapeutically effective steady state plasma levels of Compound 1 is provided by administering about 10 mg of Compound 1 or a pharmaceutically acceptable salt thereof twice a day. In further embodiments, the therapeutically effective steady state plasma levels of Compound 1 is provided by administering about 20 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 25 mg. In further embodiments, the therapeutically effective steady state plasma levels of Compound 1 is provided by administering about 25 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 30 mg. In further embodiments, the therapeutically effective steady state plasma levels of Compound 1 is provided by administering about 15 mg of Compound 1 or a pharmaceutically acceptable salt thereof twice a day. In further embodiments, the therapeutically effective steady state plasma levels of Compound 1 is provided by administering about 30 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 35 mg. In further embodiments, the therapeutically effective steady state plasma levels of Compound 1 is provided by administering about 35 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 40 mg. In further embodiments, the therapeutically effective steady state plasma levels of Compound 1 is provided by administering about 20 mg of Compound 1 or a pharmaceutically acceptable salt thereof twice a day. In further embodiments, the therapeutically effective steady state plasma levels of Compound 1 is provided by administering about 40 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 45 mg. In further embodiments, the therapeutically effective steady state plasma levels of Compound 1 is provided by administering about 45 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 50 mg. In further embodiments, the therapeutically effective steady state plasma levels of Compound 1 is provided by administering about 25 mg of Compound 1 or a pharmaceutically acceptable salt thereof twice a day. In further embodiments, the therapeutically effective steady state plasma levels of Compound 1 is provided by administering about 50 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 55 mg. In further embodiments, the therapeutically effective steady state plasma levels of Compound 1 is provided by administering about 55 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 60 mg. In further embodiments, the therapeutically effective steady state plasma levels of Compound 1 is provided by administering about 30 mg of Compound 1 or a pharmaceutically acceptable salt thereof twice a day. In further embodiments, the therapeutically effective steady state plasma levels of Compound 1 is provided by administering about 60 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 65 mg. In further embodiments, the therapeutically effective steady state plasma levels of Compound 1 is provided by administering about 65 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 70 mg. In further embodiments, the therapeutically effective steady state plasma levels of Compound 1 is provided by administering about 35 mg of Compound 1 or a pharmaceutically acceptable salt thereof twice a day. In further embodiments, the therapeutically effective steady state plasma levels of Compound 1 is provided by administering about 70 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 75 mg. In further embodiments, the therapeutically effective steady state plasma levels of Compound 1 is provided by administering about 75 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 80 mg. In further embodiments, the therapeutically effective steady state plasma levels of Compound 1 is provided by administering about 40 mg of Compound 1 or a pharmaceutically acceptable salt thereof twice a day. In further embodiments, the therapeutically effective steady state plasma levels of Compound 1 is provided by administering about 80 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 85 mg. In further embodiments, the therapeutically effective steady state plasma levels of Compound 1 is provided by administering about 85 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 90 mg. In further embodiments, the therapeutically effective steady state plasma levels of Compound 1 is provided by administering about 45 mg of Compound 1 or a pharmaceutically acceptable salt thereof twice a day. In further embodiments, the therapeutically effective steady state plasma levels of Compound 1 is provided by administering about 90 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 95 mg. In further embodiments, the therapeutically effective steady state plasma levels of Compound 1 is provided by administering about 95 mg of Compound 1 or a pharmaceutically acceptable salt thereof once a day.

In certain embodiments, the therapeutically effective steady state plasma levels of Compound 1 is provided by administering a daily dose of Compound 1 or a pharmaceutically acceptable salt thereof of about 100 mg. In further embodiments, the therapeutically effective steady state plasma levels of Compound 1 is provided by administering about 50 mg of Compound 1 or a pharmaceutically acceptable salt thereof twice a day. In further embodiments, the therapeutically effective steady state plasma levels of Compound 1 is provided by administering about 100 mg of Compound 1 or a pharmaceutically acceptable salt thereof once a day.

In some embodiments, the present methods provide mean steady state AUC₀₋₂₄ₕ (expressed in terms of ng*hr/mL) levels of Compound 1 that correlate to one or more statistically significant therapeutic effects. In certain embodiments, the therapeutically effective mean steady state AUC₀₋₂₄ₕ levels of Compound 1 provided by the methods of the present invention range from about 50 ng*hr/mL to about 2300 ng*hr/mL, including about 50 ng*hr/mL, 100 ng*hr/mL, 150 ng*hr/mL, 200 ng*hr/mL, 250 ng*hr/mL, 300 ng*hr/mL, about 400 ng*hr/mL, about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, about 1200 ng*hr/mL, about 1300 ng*hr/mL, about 1400 ng*hr/mL, about 1500 ng*hr/mL, about 1600 ng*hr/mL, about 1700 ng*hr/mL, about 1800 ng*hr/mL, about 1900 ng*hr/mL, about 2000 ng*hr/mL, about 2100 ng*hr/mL, about 2200 ng*hr/mL and about 2300 ng*hr/mL, including all ranges there between. In certain embodiments, the therapeutically effective mean steady state AUC₀₋₂₄ₕ levels of Compound 1 provided by the methods of the present invention range from about 500 ng*hr/mL to about 1000 ng*hr/mL, including about 550 ng*hr/mL, about 600 ng*hr/mL, about 650 ng*hr/mL, about 700 ng*hr/mL, about 750 ng*hr/mL, about 800 ng*hr/mL, about 850 ng*hr/mL and about 900 ng*hr/mL, including all ranges there between. In certain embodiments, the therapeutically effective mean steady state AUC₀₋₂₄ₕ levels of Compound 1 provided by the methods of the present invention range from about 600 ng*hr/mL to about 900 ng*hr/mL.

In certain embodiments, the mean steady state AUC₀₋₂₄ₕ levels of Compound 1 is provided by administering a daily dose of Compound 1 or a pharmaceutically acceptable salt thereof of about 15 mg. In further embodiments, the mean steady state AUC₀₋₂₄ₕ levels of Compound 1 is provided by administering about 15 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 20 mg. In further embodiments, the mean steady state AUC₀₋₂₄ₕ levels of Compound 1 is provided by administering about 10 mg of Compound 1 or a pharmaceutically acceptable salt thereof twice a day. In further embodiments, the mean steady state AUC₀₋₂₄ₕ levels of Compound 1 is provided by administering about 20 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 25 mg. In further embodiments, the mean steady state AUC₀₋₂₄ₕ levels of Compound 1 is provided by administering about 25 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 30 mg. In further embodiments, the mean steady state AUC₀₋₂₄ₕ levels of Compound 1 is provided by administering about 15 mg of Compound 1 or a pharmaceutically acceptable salt thereof twice a day. In further embodiments, the mean steady state AUC₀₋₂₄ₕ levels of Compound 1 is provided by administering about 30 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 35 mg. In further embodiments, the mean steady state AUC₀₋₂₄ₕ levels of Compound 1 is provided by administering about 35 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 40 mg. In further embodiments, the mean steady state AUC₀₋₂₄ₕ levels of Compound 1 is provided by administering about 20 mg of Compound 1 or a pharmaceutically acceptable salt thereof twice a day. In further embodiments, the mean steady state AUC₀₋₂₄ₕ levels of Compound 1 is provided by administering about 40 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 45 mg. In further embodiments, the mean steady state AUC₀₋₂₄ₕ levels of Compound 1 is provided by administering about 45 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 50 mg. In further embodiments, the mean steady state AUC₀₋₂₄ₕ levels of Compound 1 is provided by administering about 25 mg of Compound 1 or a pharmaceutically acceptable salt thereof twice a day. In further embodiments, the mean steady state AUC₀₋₂₄ₕ levels of Compound 1 is provided by administering about 50 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 55 mg. In further embodiments, the mean steady state AUC₀₋₂₄ₕ levels of Compound 1 is provided by administering about 55 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 60 mg. In further embodiments, the mean steady state AUC₀₋₂₄ₕ levels of Compound 1 is provided by administering about 30 mg of Compound 1 or a pharmaceutically acceptable salt thereof twice a day. In further embodiments, the mean steady state AUC₀₋₂₄ₕ levels of Compound 1 is provided by administering about 60 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 65 mg. In further embodiments, the mean steady state AUC₀₋₂₄ₕ levels of Compound 1 is provided by administering about 65 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 70 mg. In further embodiments, the mean steady state AUC₀₋₂₄ₕ levels of Compound 1 is provided by administering about 35 mg of Compound 1 or a pharmaceutically acceptable salt thereof twice a day. In further embodiments, the mean steady state AUC₀₋₂₄ₕ levels of Compound 1 is provided by administering about 70 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 75 mg. In further embodiments, the mean steady state AUC₀₋₂₄ₕ levels of Compound 1 is provided by administering about 75 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 80 mg. In further embodiments, the mean steady state AUC₀₋₂₄ₕ levels of Compound 1 is provided by administering about 40 mg of Compound 1 or a pharmaceutically acceptable salt thereof twice a day. In further embodiments, the mean steady state AUC₀₋₂₄ₕ levels of Compound 1 is provided by administering about 80 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 85 mg. In further embodiments, the mean steady state AUC₀₋₂₄ₕ levels of Compound 1 is provided by administering about 85 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 90 mg. In further embodiments, the mean steady state AUC₀₋₂₄ₕ levels of Compound 1 is provided by administering about 45 mg of Compound 1 or a pharmaceutically acceptable salt thereof twice a day. In further embodiments, the mean steady state AUC₀₋₂₄ₕ levels of Compound 1 is provided by administering about 90 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 95 mg. In further embodiments, the mean steady state AUC₀₋₂₄ₕ levels of Compound 1 is provided by administering about 95 mg of Compound 1 or a pharmaceutically acceptable salt thereof once a day.

In certain embodiments, the mean steady state AUC₀₋₂₄ₕ levels of Compound 1 is provided by administering a daily dose of Compound 1 or a pharmaceutically acceptable salt thereof of about 100 mg. In further embodiments, the mean steady state AUC₀₋₂₄ₕ levels of Compound 1 is provided by administering about 50 mg of Compound 1 or a pharmaceutically acceptable salt thereof twice a day. In further embodiments, the mean steady state AUC₀₋₂₄ₕ levels of Compound 1 is provided by administering about 100 mg of Compound 1 or a pharmaceutically acceptable salt thereof once a day.

In some embodiments, the present methods provide steady state plasma Cmax levels (or mean steady state plasma Cmax levels) of Compound 1 that correlate to one or more statistically significant therapeutic effects. In certain embodiments, the therapeutically effective steady state plasma Cmax levels (or mean steady state plasma Cmax levels) of Compound 1 provided by the methods of the present invention range from about 5 ng/mL to about 500 ng/mL, including about 5 ng/mL, 10 ng/mL, 20 ng/mL, 30 ng/mL, 40 ng/mL, 50 ng/mL, 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL about 380 ng/mL, about 390 ng/mL, about 400 ng/mL, about 410 ng/mL, about 420 ng/mL, about 430 ng/mL, about 440 ng/mL, about 150 ng/mL, about 460 ng/mL, about 470 ng/mL about 480 ng/mL, about 490 ng/mL, about 500 ng/mL, about 510 ng/mL about 520 ng/mL, about 530 ng/mL, about 540 ng/mL, about 550 ng/mL, about 560 ng/mL, about 570 ng/mL about 580 ng/mL, about 590 ng/mL and about 600 ng/mL, including all ranges there between. In some embodiments, the therapeutically effective steady state plasma Cmax levels (or mean steady state plasma Cmax levels) of Compound 1 provided by the methods of the present invention are from about 100 ng/mL to about 275 ng/mL, including about 110 ng/mL, about 120 ng/mL, about 130 ng/mL, about 140 ng/mL, about 150 ng/mL, about 160 ng/mL, about 170 ng/mL about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL, including all ranges there between. In some embodiments, the therapeutically effective steady state plasma Cmax levels (or mean steady state plasma Cmax levels) of Compound 1 provided by the methods of the present invention are from about 125 ng/mL to about 250 ng/mL.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 15 mg. In further embodiments, the therapeutically effective steady state plasma Cmax levels (or mean steady state plasma Cmax levels) of Compound 1 is provided by administering about 15 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 20 mg. In further embodiments, the therapeutically effective steady state plasma Cmax levels (or mean steady state plasma Cmax levels) of Compound 1 is provided by administering about 10 mg of Compound 1 or a pharmaceutically acceptable salt thereof twice a day. In further embodiments, the therapeutically effective steady state Cmax plasma levels (or mean steady state plasma Cmax levels) of Compound 1 is provided by administering about 20 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 25 mg. In further embodiments, the therapeutically effective steady state plasma Cmax levels (or mean steady state plasma Cmax levels) of Compound 1 is provided by administering about 25 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 30 mg. In further embodiments, the therapeutically effective steady state plasma Cmax levels (or mean steady state plasma Cmax levels) of Compound 1 is provided by administering about 15 mg of Compound 1 or a pharmaceutically acceptable salt thereof twice a day. In further embodiments, the therapeutically effective steady state Cmax plasma levels (or mean steady state plasma Cmax levels) of Compound 1 is provided by administering about 30 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 35 mg. In further embodiments, the therapeutically effective steady state plasma Cmax levels (or mean steady state plasma Cmax levels) of Compound 1 is provided by administering about 35 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 40 mg. In further embodiments, the therapeutically effective steady state plasma Cmax levels (or mean steady state plasma Cmax levels) of Compound 1 is provided by administering about 20 mg of Compound 1 or a pharmaceutically acceptable salt thereof twice a day. In further embodiments, the therapeutically effective steady state Cmax plasma levels (or mean steady state plasma Cmax levels) of Compound 1 is provided by administering about 40 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 45 mg. In further embodiments, the therapeutically effective steady state plasma Cmax levels (or mean steady state plasma Cmax levels) of Compound 1 is provided by administering about 45 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 50 mg. In further embodiments, the therapeutically effective steady state plasma Cmax levels (or mean steady state plasma Cmax levels) of Compound 1 is provided by administering about 25 mg of Compound 1 or a pharmaceutically acceptable salt thereof twice a day. In further embodiments, the therapeutically effective steady state Cmax plasma levels (or mean steady state plasma Cmax levels) of Compound 1 is provided by administering about 50 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 55 mg. In further embodiments, the therapeutically effective steady state plasma Cmax levels (or mean steady state plasma Cmax levels) of Compound 1 is provided by administering about 55 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 60 mg. In further embodiments, the therapeutically effective steady state plasma Cmax levels (or mean steady state plasma Cmax levels) of Compound 1 is provided by administering about 30 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 60 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 65 mg. In further embodiments, the therapeutically effective steady state plasma Cmax levels (or mean steady state plasma Cmax levels) of Compound 1 is provided by administering about 65 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 70 mg. In further embodiments, the therapeutically effective steady state plasma Cmax levels (or mean steady state plasma Cmax levels) of Compound 1 is provided by administering about 35 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 70 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 75 mg. In further embodiments, the therapeutically effective steady state plasma Cmax levels (or mean steady state plasma Cmax levels) of Compound 1 is provided by administering about 75 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 80 mg. In further embodiments, the therapeutically effective steady state plasma Cmax levels (or mean steady state plasma Cmax levels) of Compound 1 is provided by administering about 40 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 80 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 85 mg. In further embodiments, the therapeutically effective steady state plasma Cmax levels (or mean steady state plasma Cmax levels) of Compound 1 is provided by administering about 85 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 90 mg. In further embodiments, the therapeutically effective steady state plasma Cmax levels (or mean steady state plasma Cmax levels) of Compound 1 is provided by administering about 45 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 90 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 95 mg. In further embodiments, the therapeutically effective steady state plasma Cmax levels (or mean steady state plasma Cmax levels) of Compound 1 is provided by administering about 95 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 100 mg. In further embodiments, the therapeutically effective steady state plasma Cmax levels (or mean steady state plasma Cmax levels) of Compound 1 is provided by administering about 50 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 100 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 105 mg. In further embodiments, the therapeutically effective steady state plasma Cmax levels (or mean steady state plasma Cmax levels) of Compound 1 is provided by administering about 105 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 110 mg. In further embodiments, the therapeutically effective steady state plasma Cmax levels (or mean steady state plasma Cmax levels) of Compound 1 is provided by administering about 55 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 110 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 115 mg. In further embodiments, the therapeutically effective steady state plasma Cmax levels (or mean steady state plasma Cmax levels) of Compound 1 is provided by administering about 115 mg of Compound 1 or a pharmaceutically acceptable salt thereof once a day.

In certain embodiments, the therapeutically effective steady state plasma Cmax levels (or mean steady state plasma Cmax levels) of Compound 1 is provided by administering a daily dose of Compound 1 or a pharmaceutically acceptable salt thereof is about 120 mg. In further embodiments, the therapeutically effective steady state plasma Cmax levels (or mean steady state plasma Cmax levels) of Compound 1 is provided by administering about 60 mg of Compound 1 or a pharmaceutically acceptable salt thereof twice a day. In further embodiments, the therapeutically effective steady state plasma Cmax levels (or mean steady state plasma Cmax levels) of Compound 1 is provided by administering about 120 mg of Compound 1 or a pharmaceutically acceptable salt thereof once a day.

In some embodiments, the present methods provide steady state plasma Cmax levels (or mean steady state plasma Cmax levels) of Compound 1 that do not exceed 500 ng/mL. In certain embodiments, the therapeutically effective steady state plasma Cmax levels of Compound 1 provided by the methods of the present invention do not exceed about 500 ng/mL, including less than about 500 ng/mL, less than about 475 ng/mL, less than about 450 ng/mL, than about 425 ng/mL, less than about 400 ng/mL, less than about 375 ng/mL, than about 350 ng/mL, less than about 325 ng/mL, and less than about 300 ng/mL.

### Methods of Treating Substance Abuse Use Disorder

In some embodiments, the present disclosure provides methods of treating a substance abuse disorder (such as opioid use disorder) comprising administering an effective amount of Compound 1 or a pharmaceutically acceptable salt thereof. In some embodiments, the present methods employ Compound 1, or a pharmaceutically acceptable salt thereof, as the sole active ingredient used to treat a substance abuse disorder. In some embodiments, the present methods employ Compound 1, or a pharmaceutically acceptable salt thereof, in conjunction with one or more active ingredients used to treat a substance abuse disorder. In some embodiments, Compound 1 or a pharmaceutically acceptable salt thereof is administered in combination with an additional active ingredient used to treat a substance abuse disorder, e.g., co-formulated or administered separately.

Opioid use disorder includes signs and symptoms that reflect compulsive, prolonged self-administration of opioid substances that are used for no legitimate medical purpose or, if another medical condition is present that requires opioid treatment, that are used in doses greatly in excess of the amount needed for that medical condition. For example, an individual prescribed analgesic opioids for pain relief at adequate dosing will use significantly more than prescribed and not only because of persistent pain.

Substance abuse disorder, including cocaine, alcohol, and opioids has been associated with the dopamine reward pathways (Ross, S. & Peselow, E. The Neurobiology of Addictive Disorders. Clin Neuropharmacol 32, 269-276 (2009). The neurotransmitter GABA suppresses striatal dopamine release and blunts cocaine-induced increases in extracellular dopamine in the striatum and nucleus accumbens in animals (Dewey, S. et al. GABAergic inhibition of endogenous dopamine release measured in vivo with 11C-raclopride and positron emission tomography. J Neurosci 12, 3773-3780 (1992). Progesterone treatment has been shown to decrease craving to cocaine in clinical studies, potentially due to the GABAergic effect GABA-A positive allosteric modulator neuroactive steroids synthesized from progesterone (Sinha, R. et al. Sex steroid hormones, stress response, and drug craving in cocaine-dependent women: Implications for relapse susceptibility. Exp Clin Psychopharm 15, 445 (2007).

Stress is an important factor in the development of substance use disorders and in perpetuating the cycle of drug use, abstinence, and relapse in addicted individuals.

Progesterone treatment has been shown to decrease craving to cocaine in clinical studies, potentially due to the GABAergic effect GABA-A positive allosteric modulator neuroactive steroids synthesized from progesterone.

In some embodiments, the present disclosure provides methods of treating opioid use disorder comprising administering an effective amount of Compound 1 or a pharmaceutically acceptable salt thereof. In some embodiments, the Compound 1 is administered as a monotherapy. In some embodiments, the Compound 1 is administered as an adjunctive to the patient's existing therapy (e.g., the current standard of care). In certain embodiments, the Compound 1 is administered as an adjunctive to methadone. In certain embodiments, the Compound 1 is administered as an adjunctive to buprenorphine.

In some embodiments, after said treatment the patient experiences a substantial reduction of opioid use disorder that is characterized by an abstinence to opioid use during the period of Compound 1 administration. As used herein “abstinence to opioid use” means a negative urine drug test and no self-reported opioid use on the timeline follow-back (TLFB) survey during the period of Compound 1 administration. TLFB survey use calendars and daily recall of substance use on specific days to record quantity or frequency of opioid use. Omission of any of these criteria resulted in failure to confirm abstinence for the week.

In some embodiments, after said treatment the patient experiences a substantial reduction of opioid use disorder that is characterized by a statistically significant decrease in the percentage of opioid-free weeks in an Compound 1 treated group compared to a placebo treated group during the period of Compound 1 administration (i.e., there is a significant statistical difference between the percentage of opioid-free weeks of Compound 1 treatment relative to placebo treatment).

In some embodiments, after said treatment the patient experiences a substantial reduction of opioid use disorder that is characterized by substantial improvement is demonstrated by craving assessment—weekly self-report visual analogue scale (VAS) of need for opioids (scale 0-100, 0=not at all; 100=very much so). Response defined by significant statistical difference in the mean change in VAS score from baseline of treatment group relative to placebo (Krupitsky, E. et al. Injectable extended-release naltrexone for opioid dependence: a double-blind, placebo-controlled, multicentre randomised trial. Lancet 377, 1506-1513 (2006).

In some embodiments, after said treatment the patient experiences a substantial reduction of opioid use disorder that is characterized by a statistically significant change in retention assessment compared to a placebo treated group. As used herein, “retention assessment” means the number of days of retention on either cognitive behavioral therapy or pharmacotherapy by TLFB during the period of Compound 1 administration.

In certain embodiments, after said treatment the patient experiences a statistically significant change in retention assessment that is characterized by significant statistical difference in the mean change in number of days of retention in Compound 1 treatment group relative to placebo.

In some embodiments, the present disclosure provides methods of treating cocaine use disorder comprising administering an effective amount of Compound 1 or a pharmaceutically acceptable salt thereof. In some embodiments, the Compound 1 is administered as a monotherapy. In some embodiments, the Compound 1 is administered as an adjunctive to the current standard of care. In some embodiment, the Compound 1 is administered as an adjunctive to buprenorphine. In some embodiments, the Compound 1 is administered as an adjunctive to buprenorphine and naloxone. In some embodiments, the Compound 1 is administered as an adjunctive to naltrexone. In some embodiments, the Compound 1 administered as an adjunctive to lofexidine.

In some embodiments, the present disclosure provides methods of treating alcohol use disorder comprising administering an effective amount of Compound 1 or a pharmaceutically acceptable salt thereof. In some embodiments, the Compound 1 is administered as a monotherapy. In some embodiments, the Compound 1 is administered as an adjunctive to the current standard of care. In some embodiments, the Compound 1 is administered as an adjunctive to a benzodiazepine.

In some embodiments, the present disclosure provides methods of treating benzodiazepine use disorder comprising administering an effective amount of Compound 1 or a pharmaceutically acceptable salt thereof. In some embodiments, the Compound 1 is administered as a monotherapy. In some embodiments, the Compound 1 is administered as an adjunctive to the current standard of care. In some embodiments, the Compound 1 is administered as an adjunctive to medically supervised withdrawal (detoxification). In some embodiments, the Compound 1 is administered as an adjunctive to residential rehabilitation treatment. In some embodiments, the Compound 1 is administered as an adjunctive to mutual help groups. In some embodiments, the Compound 1 is administered as an adjunctive to outpatient substance use disorder services (e.g., counseling or medication for addiction).

### EXAMPLES

The present invention is further illustrated by reference to the following Examples. However, it is noted that these Examples, like the embodiments described above, are illustrative and are not to be construed as restricting the scope of the invention in any way.

### Example 1

Healthy subjects aged 18 to 55 years were treated with an oral suspension of Compound 1 to study the safety, tolerability, pharmacokinetics and pharmacodynamics of Compound 1 in healthy subjects. Dose and dose frequencies were evaluated in order to select a regimen that is suitable for subjects with MDD. From the results of the study, oral Compound 1 will be assessed for its potential to reduce the symptoms of MDD in a dose-dependent manner.

Study Design

The study was a randomized, double-blind, placebo-controlled multiple escalating dose study comprised of 3 cohorts that each received an oral suspension. Each cohort consisted of two groups: one group treated with Compound 1 and another treated with placebo. In each cohort, the ratio of Compound 1-treated subjects to placebo-treated subjects was 3:1.

The Compound 1-treated subjects of Cohort 1 were treated with 15.0 mg of Compound 1 once per day (QD). The Compound 1-treated subjects of Cohort 2 were treated with 30.0 mg of Compound 1 QD. The Compound 1-treated subjects of Cohort 3 were treated with 60.0 mg of Compound 1 QD.

A Food Effect Cohort (Cohort 4) was conducted to assess the effect of food on the PK profile of a single dose of Compound 1 when administered to healthy subjects. The subjects of Cohort 4 were treated with 30 mg of Compound 1 QD.

Dosing:

Patients in each cohort were treated with Compound 1 for 14 consecutive days, unless dosing was halted by the Safety Review Committee (SRC). The dosing of subjects in each of the cohorts was staggered with the decision to dose escalate based on SRC review of a minimum of 14 days of observation of safety and tolerability data and review of the available plasma PK data from the preceding cohort(s). Thus, dose escalation was predicated on tolerability of the prior cohorts.

Compound 1 was administered under fasted conditions (no food or drink, except water, for at least 10 hours prior to dosing). Immediately after administration of Compound 1, the subject was be administered 240 mL water. No additional fluid intake was allowed until 1 hour after Compound 1 administration.

Cohort 1 subjects received a single 15.0 mg dose of a Compound 1 suspension on the morning of Days 1-14. Cohort 2 subjects received a single 30.0 mg dose of a Compound 1 suspension on the morning of Days 1-14. Cohort 3 subjects received a single 60.0 mg dose of a Compound 1 suspension on the morning of Days 1-14. For all cohorts, the last treatment was administered on the morning of Day 14.

Cohort 4 subjects received a single 30 mg dose of a Compound 1 suspension on Days 1 and 5. The Day 1 dose was administered after a minimum of 10 hour fasting. No additional fluid intake was allowed until 1 hour after drug administration. A standard meal was given at least 4 hours post-dose. The Day 5 dose was administered following a high-fat, high calorie meal. Participants remained at the clinical site for a total of 8 days to complete PK sampling after the second dose.

Blood and urine were obtained during each treatment period at designated times for PK and other analyses (see below). Standard safety assessments were measured during each treatment period.

Pharmacokinetic (PK) Assessments

PK parameters (e.g., Cₘₐₓ, Tₘₐₓ, T_{1/2}, AUC, etc.) for healthy patients in each cohort was compared to assess the suitability of Compound 1 suspension for the treatment of MDD. Data were obtained from the blood plasma samples collected from each cohort according to the schedule provided.

Plasma samples were analyzed to determine Compound 1 concentrations using a validated assay method. Pharmacokinetic variables (including but not limited to Cₘₐₓ, Tₘₐₓ and AUC₍₀₋ₗₐₛₜ₎), were calculated using non-compartmental analysis. PK parameters for Compound 1 were derived from the plasma concentration data using non-compartmental analysis with Phoenix™ WinNonlin® v 8.0 (Pharsight Corporation, USA).

Protocol:

Blood (Cohorts 1-3):

For each cohort, blood samples were collected on Days 1, 2, 3, 4, 5, 6 and 14 at the following time points: Day 1 at pre-dose (0 hour), 0.25, 0.5, 0.75, 1, 1.5, 2, 2.5, 3, 4, 6, 8, 12, 16 h post-dose; Day 2 at pre-dose (24 h), Day 3 at pre-dose (48 h), Day 4 at pre-dose (72 h), Day 5 at pre-dose (96 h), Day 6 at pre-dose (120 h); Day 14 at pre-dose 0.25, 0.5, 0.75, 1, 1.5, 2, 2.5, 3, 4, 6, 8, 12, 16, 24, 36, 48, and 72 hours. Trough level blood samples were collected on Days 2, 3, 4, 5, 6 and 14, prior to the morning dose administrations.

The following PK parameters were calculated based on the plasma concentrations of Compound 1: maximum observed concentration (Cmax) on Day 1 and at steady state on Day 15 (Cmax,SS), Time of Cmax (Tmax) and Cmax,SS (Tmax,SS), area under the concentration-time curve through the dosing interval on Day 1 and 15 (AUCtau and AUCSS), total clearance at steady state, measured on Day 15 (CLSS), and volume of distribution at steady state, measured on Day 15 (VSS).

Urine (Cohorts 1-3):

Urine was collected/pooled at the following collection windows: Day −1 (6 hours) and at Day 14: (0 to 6 hours), (6 to 12 hours), (12 to 24 hours), and (24-48 hours). Urine samples were analyzed to determine Compound 1 concentrations using a validated assay method. Pooling of urine across patients may be allowed if volumes are not sufficient to allow individual determination.

The following PK parameters were calculated based on the urine concentrations of Compound 1: absolute and cumulative amount of Compound 1 excreted in urine and renal clearance (CLR).

Blood (Cohort 4):

Serial blood samples were collected relative to the dosing of PRAX-114 at the following time points on both Day 1 and Day 5: Pre-dose (0 hours), 0.25, 0.50, 0.75, 1.00, 1.50, 2.00, 2.50, 3.00, 4.00, 6.00, 8.00, 12.00, 16.00, 24.00, 36.00, 48.00 and 72.00 hours post-dose (±2 min). Urine (Cohort 4): No urine analysis was conducted in Cohort 4.

Pharmacodynamic Assessment

Pharmacodynamic (PD) effects of first dose and steady state Compound 1 concentrations on wake electroencephalograms (EEGs) was studied. Standard 16 channel continuous EEGs were obtained at the following time points: Day-1, Day 1 (1 h after dosing), and Day 14 (1 h after dosing).

Safety Assessments/Monitoring

Adverse events (AEs) were monitored throughout the duration of the study.

To monitor for possible adverse events, vital signs, hematology and clinical chemistry laboratory parameters, ECG readings, neurological examination findings, and EEG parameters and abnormal findings were recorded at each visit.

Statistical Analysis

Descriptive statistics were calculated for plasma and urine PK parameter and concentration data, and summarized by study day and time point. Arithmetic means, coefficient of variation (CV), standard deviation, median, minimum, and maximum values, and number of observations were calculated for all PK parameters and trough concentration data. Except for Tmax, the geometric mean, geometric standard deviation, and geometric CV were provided for all PK parameter and concentration data.

Cohorts 1-3 Results:

The following pharmacokinetic parameters were determined for each cohort: maximum plasma concentration (Cₘₐₓ, observed, Day 1 only); time to reach the maximum plasma concentration (Tₘₐₓ, observed, Day 1 only); and area under the plasma concentration-time curve from time 0 to 24 h post dose (AUC₀₋ₜₐᵤ).

The following steady state pharmacokinetic (Day 14) parameters were determined: t_{1/2}: elimination half-life associated with the terminal slope (λz) of the semi-logarithmic drug concentration-time curve, calculated as 0.693/λz; C_{max,ss}: maximum plasma concentration (observed); T_{max,ss}: time to reach the maximum plasma concentration (observed); AUCₛₛ: area under the plasma concentration-time curve from 0 to 24 h post dose; AUC_{inf}: area under the plasma concentration-time curve from 0 to infinite time; C_{avg}: average concentration over dosing interval; CLss/F: steady state clearance; and Vz/F: volume of distribution of terminal phase.

Table 1 shows a summary of the observed PK parameters Day 1.

TABLE 1
Summary of Parameters for Compound 1 (calculated from Day 1 data for Cohorts 1-3)

| | | Tₘₐₓ | Cₘₐₓ | AUC₀₋ₜₐᵤ | Ctrough (Day 2) |
|---|---|---|---|---|---|
| Treatment | Statistic | (h) | (ng/mL) | (h*ng/mL) | (ng/mL) |
| Cohort 1 | Mean | 1.056 | 57.367 | 378.685 | 4.207 |
| | SD | 0.273 | 13.169 | 119.963 | 2.298 |
| | Min | 0.75 | 39.10 | 219.21 | 1.69 |
| | Median | 1.00 | 55.00 | 394.97 | 3.89 |
| | Max | 1.50 | 76.00 | 581.62 | 8.01 |
| | % CV | 25.9 | 23.0 | 31.7 | 54.6 |
| | Geo Mean | 1.027 | 56.016 | 361.762 | 3.716 |
| | CV % g mean | 25.08 | 23.59 | 33.22 | 56.06 |
| Cohort 2 | Mean | 2.028 | 132.511 | 842.141 | 9.353 |
| | SD | 2.323 | 50.359 | 181.943 | 3.3335 |
| | Min | 0.75 | 34.60 | 449.41 | 5.06 |
| | Median | 1.00 | 156.00 | 899.25 | 8.62 |
| | Max | 8.00 | 195.00 | 1030.39 | 14.50 |
| | CV % | 114.6 | 38.0 | 21.6 | 35.7 |
| | Geometric Mean | 1.432 | 120.018 | 820.402 | 8.805 |
| | CV % Geometric | 91.88 | 57.47 | 26.22 | 38.93 |
| | Mean | | | | |
| Cohort 3 | Mean | 1.139 | 363.889 | 2343.762 | 19.024 |
| | SD | 0.435 | 96.801 | 585.039 | 10.024 |
| | Min | 0.75 | 213.00 | 1405.82 | 5.44 |
| | Median | 1.00 | 341.00 | 2356.80 | 20.40 |
| | Max | 2.00 | 511.00 | 3128.23 | 32.30 |
| | CV % | 38.2 | 26.6 | 25.0 | 52.7 |
| | Geometric Mean | 1.074 | 352.127 | 2274.106 | 16.049 |
| | CV% Geometric | 36.72 | 28.13 | 27.16 | 75.74 |
| | Mean | | | | |

Table 2 shows a summary of the observed PK parameters Day 14 for Cohorts 1-3.

TABLE 2
Summary of Parameters for Compound 1 (calculated from Day 14 data)

| | | t_{1/2} | T_{max, ss} | C_{max, ss} | AUCinf | CLss/F | Vz/F | AUCss | C_{avg} |
|---|---|---|---|---|---|---|---|---|---|
| Treatment | Statistic | (h) | (h) | (ng/mL) | (h*ng/mL) | (L/h) | (L) | (h*ng/mL) | (ng/mL) |
| Cohort 1 | Mean | 14.772 | 1.139 | 66.333 | 623.3 | 31.415 | 655.194 | 506.288 | 21.093 |
| | SD | 2.260 | 0.435 | 13.733 | 182.7 | 8.313 | 130.993 | 129.280 | 5.387 |
| | Min | 11.48 | 0.75 | 41.50 | 351 | 19.57 | 464.09 | 306.85 | 12.79 |
| | Median | 15.26 | 1.00 | 66.00 | 595.2 | 30.41 | 656.82 | 493.56 | 20.57 |
| | Max | 17.53 | 2.00 | 91.80 | 1003 | 48.88 | 842.72 | 766.41 | 31.93 |
| | % CV | 15.3 | 38.2 | 20.7 | 29.3 | 26.5 | 20.0 | 25.5 | 25.5 |
| | Geo Mean | 14.613 | 1.074 | 65.010 | 600.228 | 30.497 | 642.968 | 491.871 | 20.497 |
| | CV % Geo Mean | 15.90 | 36.72 | 21.98 | 29.87 | 26.08 | 21.16 | 26.08 | 26.08 |
| Cohort 2 | Mean | 12.701 | 0.964 | 164.286 | 1226.440 | 30.949 | 570.566 | 1016.979 | 35.089 |
| | SD | 1.233 | 0.267 | 46.248 | 284.205 | 7.912 | 180.389 | 222.845 | 7.581 |
| | Min | 10.47 | 0.75 | 103.00 | 805.89 | 23.42 | 405.14 | 646.59 | 18.73 |
| | Median | 12.90 | 1.00 | 150.00 | 1268.36 | 30.12 | 513.72 | 995.94 | 37.47 |
| | Max | 14.19 | 1.50 | 222.00 | 1544.30 | 46.40 | 949.89 | 1280.73 | 42.93 |
| | CV % | 9.7 | 27.7 | 28.2 | 23.2 | 25.6 | 31.6 | 21.9 | 21.6 |
| | Geo Mean | 12.647 | 0.937 | 158.580 | 1196.442 | 30.178 | 550.639 | 994.095 | 34.183 |
| | CV % Geo Mean | 10.11 | 25.67 | 29.64 | 24.88 | 24.02 | 28.09 | 24.02 | 26.22 |
| Cohort 3 | Mean | 12.230 | 0.969 | 355.000 | 2373.932 | 32.142 | 574.599 | 2084.130 | 86.839 |
| | SD | 1.323 | 0.248 | 157.003 | 823.150 | 11.640 | 242.861 | 729.880 | 30.412 |
| | Min | 9.79 | 0.75 | 145.00 | 1368.69 | 17.36 | 302.08 | 1190.49 | 49.60 |
| | Median | 12.42 | 1.00 | 371.50 | 2439.64 | 28.45 | 531.34 | 2109.71 | 87.90 |
| | Max | 13.82 | 1.50 | 565.00 | 3931.46 | 50.40 | 971.28 | 3457.01 | 144.04 |
| | CV % | 10.8 | 25.6 | 44.2 | 34.7 | 36.2 | 42.3 | 35.0 | 35.0 |
| | Geo Mean | 12.164 | 0.944 | 320.410 | 2253.016 | 30.398 | 533.456 | 1973.814 | 82.242 |
| | CV % Geo Mean | 11.36 | 23.83 | 54.42 | 35.80 | 36.78 | 42.61 | 36.78 | 36.78 |

Conclusions:

Compound 1 underwent rapid absorption with an approximate proportional increase in Cₘₐₓ and AUC parameters at steady state. Where steady state data was available mean t_{1/2} was varied between 12.23 and 14.77 h and steady state clearance was determined at approximately 31 L/h.

Rapid absorption was observed with a Tₘₐₓ occurring within the first 2 h of dosing (Tables 1-2). An accumulation factor of the dosing interval (ratio of AUC_{Day 14}/AUC_{Day 1}) of approximately 1.25 for Cohort 1 and Cohort 2 (Tables 1 and 2). For Cohort 3, the accumulation factor was 0.89 (Tables 1 and 2). The mean plasma concentration (ng/mL) for the first 24 hours after the initial dose is illustrated in FIG. 1. The mean plasma concentration (ng/mL) for the 24 hours after the last dose is illustrated in FIG. 2.

Mean t½ for Cohort 1, Cohort 2 and Cohort 3 was 14.77±2.26 h, 12.70±1.23 h and 12.23±1.32 h, respectively. Steady state clearance was approximately 31 L/h for the three cohorts at Day 14 and mean Vz/F was 655, 570 and 574 L for Cohorts 1, 2 and 3, respectively. A proportional increase in Cavg, AUCss and AUCinf was observed between Cohorts 1 and 3 (Tables 1 and 2).

In Cohorts 1-3s, there were no Serious Adverse Events and no clinically relevant vital sign, ECG, or lab abnormalities. Additionally, several subjects experienced elevated mood at doses of 30 mg and 60 mg

Cohort 4 Results:

Table 3 shows a summary of the observed PK parameters Days 1 (fasted) and 5 (fed).

TABLE 3

| Compound 1 | | |
|---|---|---|
| Mean (SD) PK Parameters | | |
| Following Oral | | |
| Administration for Food | | |
| Effect Cohort Parameter | Fasted (Day 1) n = 10 | Fed (Day 1) n = 10 |
| Cₘₐₓ (ng/mL) | 84.18 (48.245) | 45.36 (10.74) |
| Tₘₐₓ (h) | 1.00 (0.75, 1.77) | 3.25 (1.00, 6.00) |
| AUC₀₋ₗₐₛₜ (h · ng/mL) | 597.7 (302.85) | 629.9 (183.63) |
| AUC_{inf} (h · ng/mL) | 602.3 (304.84) | 634.2 (185.13) |
| t _{1/2} (h) | 11.22 (1.7749) | 11.26 (0.86406) |
| AUC %_{Extrap} | 0.8702 (0.50874) | 0.6632 (0.28199) |

For Tₘₐₓ, Median (Minimum, Maximum) values are displayed.

Conclusions:

Subjects displaying high concentrations following fasted conditions also tended to show higher concentrations (relative to others within the treatment group) following fed conditions. Similarly, subjects with low Compound 1 concentrations following fasted conditions tended to have low Compound 1 concentrations following fed conditions. The t½ was similar for both fasted and fed conditions (Table 3).

There was evidence of a food effect for Compound 1 when administered as a single 30 mg dose. Approximately 15 to 20% greater overall exposure as measured by AUC0-last and AUCinf was observed following fed conditions. In addition, Cmax was 1.5 to 1.6 times higher under fasting conditions with a tendency for a shorter Tmax compared to fed conditions.

### Example 2

Patients with severe major depressive disorder (MDD) aged 18 to 65 years were treated with an oral suspension of Compound 1 to study the safety, tolerability, pharmacokinetics, and efficacy of Compound 1 in the treatment of severe MDD.

The study showed that patients receiving a daily dose of 45 mg or 80 mg of Compound 1 exhibited reduced symptoms of moderate to severe MDD.

Study Design

The study is an open-label, study comprised of two dosing periods, Part A and Part B, during which patients were treated with an oral suspension of Compound 1. Part A was an open-label assessment of two dose levels of Compound 1 (45 mg qHS Cohort 1 and 80 mg qHS Cohort, i.e., once daily administration at bedtime) administered for 7 days inpatient followed by 7 days outpatient. PRAX-114 was administered at 4 PM on Day 1 in order to collect post-dosing PK samples that could not be collected with qHS dosing.

Part B will be an assessment of a single level of Compound 1 (60 mg qHS) administered for 14 days in an outpatient setting.

Each part of the clinical trial will enroll an independent set of participants. Participants from Part A will not be eligible to enroll in Part B. Each participant will complete three periods: Screening, Treatment Period (14 days of dosing PO daily), and Safety Follow-up.

Screening Period:

The Screening Period for both Parts A and B will be up to 14 days in duration (Day −14 to Day −1). Prior to any clinical trial procedures, participants will provide written informed consent to participate in the clinical trial. Screening assessments will include: medical history, demographics, vital signs, physical examination (including height and weight), drug screen, clinical laboratory tests, an electrocardiogram (ECG), the Mini International Neuropsychiatric Interview (MINI), the Antidepressant Treatment History Questionnaire (ATRQ), HAM-D, and C-SSRS assessment.

Treatment Period:

The treatment period for both Parts A and B will be 14 days in duration.

In Part A, participants were administered daily doses of Compound 1 (45 mg Cohort and 80 mg Cohort) at 4 PM on Day 1 and qHS on all other study days for 7 days inpatient followed by 7 days outpatient (with subsequent Cohorts receiving up to 120 mg qHS). Participants checked into the clinic on Day 1. During this period the following assessments were performed: adverse event assessments, vital signs, physical examinations, blood and urine samples for safety assessments, ECGs, C-SSRS assessments, blood samples for PK, and efficacy assessments (HAM-D, MADRS, HAM-A, CGI [CGI-S and CGI-I], PSQI, and SDQ). Baseline assessments occurred on Day 1 prior to administration of study drug. On Day 8, prior to discharge, participants were supplied with sufficient Compound 1 to complete 7 days of dosing.

In Part B, participants were administered daily doses Compound 1 (60 mg qPM Cohort) for 14 days as outpatients (with subsequent Cohorts receiving up to 120 mg qPM). During this period the following assessments were performed: adverse event assessments, vital signs, physical examinations, blood and urine samples for safety assessments, ECGs, C-SSRS assessments, blood samples for PK, and efficacy assessments (HAM-D, MADRS, HAM-A, CGI [CGI-S and CGI-I], PSQI, and SDQ). Baseline assessments occurred on Day 1 prior to administration of study drug, and participants were supplied with sufficient Compound 1 to complete 7 days of dosing at that visit as well as at the Day 8 visit.

Safety Follow-Up Period

In both Parts A and B, participants will return to the clinic for safety follow-up visits on Day 15, Day 21 (±1 d), and Day 28 (±1 d). During these visits the following assessments will be performed: adverse event assessment, vital signs, physical examination, drug screen (Day 15 only), clinical laboratory tests, an ECG, C-SSRS assessment, and efficacy assessments (HAM-D, MADRS, HAM-A, CGI [CGI-S and CGI-I], SDQ, and PSQI).

Patient Population:

This trial will enroll participants with severe MDD without confounding medical or psychiatric disorders that would jeopardize the safety or scientific validity of the clinical trial. Eligibility will be confirmed by Sponsor or designee.

Number of Participants:

The clinical trial is planned to include between 24 and up to a total of 60 participants across both parts (Part A and B). In Part A, 13 participants were treated in the 45 mg Cohort and 7 participants were treated in the 80 mg Cohort. In Part B, between 10 to 12 participants are planned to be treated in the 60 mg Cohort.

Inclusion Criteria:

A participant must meet the following criteria at Screening to be eligible to participate in this clinical trial:
Males and females between the ages of 18 and 65 years (inclusive).
Weight of at least 50 kg with body mass index between 18 and 30 kg/m2 (inclusive).
Have a clinical diagnosis of severe MDD that has been present for at least a 4-week period, with a HAM-D score at screening of ≥22.
All chronic medications or interventions, especially those for depression, must have been stable for at least four weeks prior to Screening and remain stable throughout the clinical trial.
Use of a medically acceptable method of contraception throughout the duration of the clinical trial and for three months thereafter.
Willing to sign an informed consent document indicating that he/she understands the purpose of the clinical trial and the procedures that are required for the clinical trial and that he/she is willing to participate in the trial and complete all applicable assessments and comply with the protocol.

Exclusion Criteria:

A participant who meets any of the following criteria at Screening (unless otherwise specified) will be excluded from this clinical trial:
Ongoing or history of any medical or surgical condition that, in the judgment of the Investigator, might jeopardize the participant's safety or interfere with the absorption, distribution, metabolism or excretion of Compound 1.
Known hypersensitivity to any component of the formulation of Compound 1.
Taking any of the following medicines: Bupropion, Buspirone, Midazolam, Alprazolam, Nefazodone, Trazadone, Carbamazepine, Clonazepam, Quazepam, Tiagabine, Aripiprazole, olanzapine, quetiapine, brexpiprazole, and Triazolam.
Use of any experimental or investigational drug or device within 30 days prior to first dose of Compound for 5 half-lives of Compound 1, whichever is longer.
Clinically significant unstable medical or psychiatric condition, other than severe MDD and moderate to severe anxiety in the opinion of the Investigator.
Clinically significant laboratory abnormalities that in the opinion of the Investigator would jeopardize the safe conduct of the trial.
Clinically significant abnormalities in a 12-lead ECG at Screening, per the review of a cardiologist.
History of treatment resistant depression in the current episode; defined as having failed three adequate trials of antidepressant therapy with treatments from at least two different therapeutic classes for a sufficient amount of time over which a beneficial effect is generally expected as determined with the ATRQ.
History of a suicide attempt in the last 2 years.
History of bipolar disorder.
History of a psychotic episode in last 2 years, or any diagnosis of psychotic disorder such as schizophrenia, schizoaffective disorder.
History of alcohol abuse within the past two years or daily consumption of more than 4 standard alcohol-containing beverages for males or more than 2 standard alcohol-containing beverages for females.
Has ingested alcohol in the 24-hour period prior to Day 1 of the clinical trial or is unwilling to abstain from drinking alcohol throughout the treatment period.
History of substance abuse in the past 2 years or a positive drug screen. A positive cannabis screen can be repeated.
History of seizures in the past 5 years or being treated for seizures in the last 5 years.
Psychosocial or addictive disorders that would interfere with participant's ability to give informed consent or could compromise compliance with the protocol.
Any other significant disease, disorder or abnormalities, that, in the opinion of the Investigator, may either put the participant at risk because of participation in the clinical trial, may compound the result of the clinical trial, or affect the participant's ability to participate in the clinical trial.

Dosing:

Part A:

Patients in Part A were treated with Compound 1 for 14 consecutive days, unless dosing is halted by the Safety Review Committee (SRC). Patients were administered daily doses of Compound 1 for 7 days inpatient followed by 7 days outpatient.

In the 45 mg Cohort, patients were administered once daily dose of 45 mg of Compound 1 at bedtime for 7 days inpatient followed by once daily dose of 45 mg of Compound 1 at bedtime for 7 days outpatient.

In the 80 mg Cohort, patients were administered once daily dose of 80 mg of Compound 1 at bedtime for 7 days inpatient followed by once daily dose of 80 mg of Compound 1 at bedtime for 7 days outpatient.

Part B:

Patients in Part B will be treated with Compound 1 for 14 consecutive days, unless dosing is halted by the Safety Review Committee (SRC). Patients were administered daily doses of Compound 1 for 14 days as outpatients.

In the 60 mg Cohort, patients were administered once daily dose of 60 mg of Compound 1 at bedtime for 7 days inpatient followed by once daily dose of 60 mg of Compound 1 at bedtime for 7 days outpatient.

Blood and urine were obtained during each treatment period at designated times for PK and other analyses (see below). Standard safety assessments were measured during each treatment period.

Formulation

The Compound 1 drug product will be formulated as a suspension whose composition is summarized in Table 3. The Compound 1 oral suspension is planned to contain from 1 to 20 mg/mL of Compound 1.

TABLE 3
Compound 1 Composition of Drug Product
Suspension, 1 mg/mL to 20 mg/mL

| | | Amount, |
|---|---|---|
| Ingredient | Purpose | mg/mL |
| Compound 1 | Active | 1-20 mg/mL |
| Hypromellose 2910, 4000 cP, | Suspension | 5.0 |
| USP | stabilizer | |
| Poloxamer 188, USP | Dispersing agent | 5.0 |
| Purified Water, USP or higher | Excipient | q.s. |
| quality | | |
| To make | | 1.0 mL |

A placebo to match the Compound 1 oral suspension will be manufactured having substantially the same composition as the active drug product but without Compound 1 (active pharmaceutical ingredient). However, microcrystalline cellulose will be used to mimic the appearance of the suspended Compound 1. The composition of the Placebo is summarized in Table 4.

TABLE 4
Composition of Placebo Drug Product Suspension

| | | Amount, |
|---|---|---|
| Ingredient | Purpose | mg/mL |
| Microcrystalline Cellulose, NF | API simulant | 5.0-20.0* |
| (MCC) | | |
| Hypromellose 2910, 4000 cP, | Suspension | 5.0 |
| USP | stabilizer | |
| Poloxamer 188, USP | Dispersing agent | 5.0 |
| Purified water, USP or higher | Excipient | q.s. |
| quality | | |
| To make | | 1.0 mL |

Pharmacokinetic (PK) Assessments

PK parameters (e.g., Cₘₐₓ, Tₘₐₓ, occurrence of steady state, etc.) for MDD patients in each cohort will be compared to assess the suitability of Compound 1 suspension for the treatment of MDD. Data will be obtained from the blood plasma samples collected from each cohort according to the schedule provided.

Plasma samples will be analyzed to determine Compound 1 concentrations using a validated assay method. Pharmacokinetic variables (including but not limited to Cₘₐₓ, and Tₘₐₓ) will be calculated using non-compartmental analysis. PK parameters for Compound 1 will be derived from the plasma concentration data using non-compartmental analysis with Phoenix™ WinNonlin® v 8.0 (Pharsight Corporation, USA).

Protocol:

Blood:

Part A:

For Part A, blood samples were collected on Days 1, 2, 3, 4, 5, 6, 7, 15 and 28 at the following time points: Days 1-7 at about 1 hour pre-dose; Days 1-7 at about 1 hour after dosing, Day 15 and Day 28. Part B: For Part B, blood samples will be collected on Days described for Part A.

The occurrence of steady state will be assessed by visual inspection of individual participant trough concentration time course. Accumulation ratio will be estimated by comparing the Day 7 and Day 1 trough plasma concentrations of Compound 1.

Pharmacodynamic Assessment

During Parts A and B, the following clinical scores will be assessed to determine the depression symptoms experienced by subjects: total Hamilton Depression Rating Scale (HAM-D) value, Montgomery Asberg Depression Rating Scale (MADRS), Hamilton Rating Scale for Anxiety (HAM-A), Clinical Global Impression (CGI) and, in particular, the CGI-severity (CGI-S) and CGI incidence (CGI-I) subscales, Pittsburgh Sleep Quality Index (PSQI), and Symptoms of Depression Questionnaire (SDQ).

Part A:

In Part A, HAM-D, MADRS, HAM-A, CGI-S, and CGI-I values were collected in the morning of Days 1, 2, 3, 4, 5, 6, 7, 8, 15, 21 and 28. PSQI and SDQ values will be collected on Days 1, 8, 15, 21 and 28. Part B: In Part B, HAM-D, MADRS, HAM-A, CGI-S, CGI-I, PSQI and SDQ values will be collected on the days described for Part A.

HAM-D Response was defined as a reduction from baseline of ≥50% in total HAM-D score. HAM-D remission was defined as a total HAM-D score of ≤7.

Safety Assessments/Monitoring

Adverse events (AEs) will be monitored throughout the duration of the study.

To monitor for possible adverse events, vital signs, hematology and clinical chemistry laboratory parameters, ECG readings, neurological examination findings, and EEG parameters and abnormal findings were recorded at each visit.

Statistical Analysis

Efficacy Analysis:

For both parts of the clinical trial, the HAM-D total score and change from baseline values will be summarized by treatment group and time point. In addition, the change from baseline in HAM-D total score will also be analyzed using paired t-tests or similar methods. The null hypothesis of this test is that the mean difference in HAM-D total score between paired observations (i.e., pre- and post-treatment) is zero. Similar analysis methods will be used for all secondary and exploratory efficacy variables.

Pk Analysis:

Plasma concentrations of Compound 1 will be summarized using descriptive statistics by time point. The occurrence of steady state will be assessed by visual inspection of individual participant trough concentration time course. Accumulation ratio will be estimated by comparing the Day 7 and Day 1 trough plasma concentrations of Compound 1.

A validated bioanalytical method will be utilized for the determination of plasma concentrations of Compound 1. Plasma samples may also be used for additional exploratory bioanalytical method development and/or metabolite characterization purposes only.

Plasma concentrations will be summarized using descriptive statistics. If the concentration of Compound 1 is reported as below the limit of quantitation (BLQ) a value of zero will be assigned for the purposes of calculating descriptive statistics. Individual and mean concentrations will be presented as BLQ if below the bioanalytical quantitation limit.

The PK population is defined as all participants with at least one valid bioanalytical plasma concentration of Compound 1.

Part a Efficacy Results:

Efficacy (as determined by reduction Mean Change from Baseline in HAM-D score) was observed in patients that were undergoing their first course of antidepressant treatment, as well as in patients that failed previous courses of antidepressant treatment. Efficacy was observed in the absence of a background antidepressant (i.e., Compound 1 administered as monotherapy) and in presence of an antidepressant (i.e., Compound 1 administered in combination with another antidepressant.

45 mg Cohort:

On Day 7, 11 of the 13 patients met the HAM-D Response or Remission criterion. On Days 7 and 15, the least squares Mean Change from Baseline in HAM-D score was −17.8 and −13.2, respectively. On Day 15, 8 of the 13 patients met the HAM-D Response or Remission criteria.

80 mg Cohort:

On Day 7, all 7 patients met the HAM-D Response or Remission criterion. On Days 7 and 15, the least squares Mean Change from Baseline in HAM-D score was −20.0 and −16.0, respectively. On Day 15, 6 of the 7 patients met the HAM-D Response or Remission criteria.

The dose was well tolerated with no change in Adverse Event profile compared to the 45 mg Cohort.

Part a PK Results:

Using statistical modelling based upon the PK studies conducted in Example 1, the following PK parameters that correlate to the reduction in HAM-D score observed in the 45 mg and 80 mg Cohorts were predicted:

| Cohort | Model | Cmax (ng/mL | AUC₀₋₂₄ (h · ng/mL) |
|---|---|---|---|
| 45 mg | Model 1 | 47.2-175 | 341-1420 |
| | Model 2 | 32-129 | 337-1410 |
| 80 mg | Model 1 | 83.9-312 | 606-2530 |
| | Model 2 | 56.9-229 | 599-2500 |
| | Model 3 | 150-556 | 1080-4510 |

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

### EMBODIMENTS

1. A method of treating depression in a patient in need thereof comprising orally administering a daily dose of about 5 mg to about 120 mg of Compound 1:

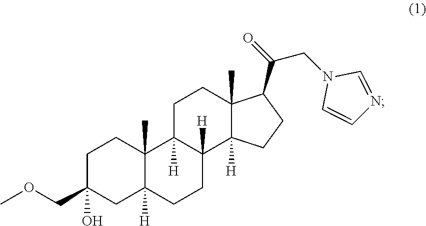

or a pharmaceutically acceptable salt thereof, wherein the depression is selected from the group consisting of major depressive disorder, major depressive disorder with suicidal risk, clinical depression, postnatal or postpartum depression, treatment resistant postpartum depression, perimenopausal depression, menopausal depression, child and adolescent depression, premenstrual dysphoric disorder (PMDD), atypical depression, melancholic depression, Psychotic Major Depression (PMD), catatonic depression, Seasonal Affective Disorder (SAD), persistent depressive disorder (dysthymia), double depression, Depressive Personality Disorder (DPD), Recurrent Brief Depression (RBD), minor depressive disorder, bipolar disorder or manic depressive disorder, bipolar depression with suicidal risk, post-traumatic stress disorders, depression caused by chronic medical conditions, depressive disorder due to another medical condition, treatment-resistant depression, refractory depression, substance/medication induced depressive disorder, depression with anxiety, suicidality, suicidal ideation, or suicidal behavior.
2. The method of embodiment 1, wherein the depression is major depressive disorder (MDD).
3. The method of embodiment 2, wherein the MDD patient is a patient with insomnia.
4. The method of any one of embodiments 1-3, wherein major depressive disorder is severe major depressive disorder.
5. The method of any one of embodiments 1-4, wherein prior to said treatment, the patient's total Hamilton Depression Rating Scale (HAM-D) value is at least 22.
6. The method of embodiment 1, wherein the depression is treatment resistant depression.
7. The method of any one of embodiments 1-6, wherein about 15 mg to about 120 mg of Compound 1 or a pharmaceutically acceptable salt thereof is administered per day.
8. The method of any one of embodiments 1-7, wherein about 15 mg of Compound 1 or a pharmaceutically acceptable salt thereof is administered once a day.
9. The method of any one of embodiments 1-7, wherein about 15 mg of Compound 1 or a pharmaceutically acceptable salt thereof is administered twice a day.
10. The method of any one of embodiments 1-7, wherein about 30 mg of Compound 1 or a pharmaceutically acceptable salt thereof is administered once a day.
11. The method of any one of embodiments 1-7, wherein about 30 mg of Compound 1 or a pharmaceutically acceptable salt thereof is administered twice a day.
12. The method of any one of embodiments 1-7, wherein about 60 mg of Compound 1 or a pharmaceutically acceptable salt thereof is administered once a day.
12(a). The method of any one of The method of any one of embodiments 1-7, wherein about 30 mg of Compound 1 or a pharmaceutically acceptable salt thereof is administered twice a day.
12(b). The method of any one of The method of any one of embodiments 1-7, wherein about 80 mg of Compound 1 or a pharmaceutically acceptable salt thereof is administered once a day.
12(c). The method of any one of The method of any one of embodiments 1-7, wherein about 40 mg of Compound 1 or a pharmaceutically acceptable salt thereof is administered twice a day.
12(d).The method of any one of The method of any one of embodiments 1-7, wherein about 100 mg of Compound 1 or a pharmaceutically acceptable salt thereof is administered once a day.
12(e). The method of any one of The method of any one of embodiments 1-7, wherein about 50 mg of Compound 1 or a pharmaceutically acceptable salt thereof is administered twice a day.
12(f).The method of any one of The method of any one of embodiments 1-7, wherein about 120 mg of Compound 1 or a pharmaceutically acceptable salt thereof is administered once a day.
12(g). The method of any one of The method of any one of embodiments 1-7, wherein about 60 mg of Compound 1 or a pharmaceutically acceptable salt thereof is administered twice a day.
13. The method of any one of embodiments 1-12, wherein said administering is for about 2 weeks, about 4 weeks, about 8 weeks, about 10 weeks, about 12 weeks, about 24 weeks or about 50 weeks.
14. The method of any one of embodiments 1-6 and 13, wherein the administering comprises:
   (a) administering Compound 1 or a pharmaceutically acceptable salt thereof for at least one week at an initial daily dose and
   (b) administering Compound 1 or a pharmaceutically acceptable salt thereof for at least one week at a maintenance daily dose, wherein the initial daily dose is greater than the maintenance daily dose.
15. The method of any one of embodiments 1-14, further comprising titrating the dose of Compound 1 or a pharmaceutically acceptable salt thereof for at least one week until a steady state is achieved in the patient.
16. The method of any one of embodiments 1-15, wherein after said administering for a period of at least 1 week, the patient experiences a substantial reduction in depression compared to prior to said administering.
17. The method of any one of embodiments 1-16, wherein after said administering for a period of at least 1 week, the patient experiences a reduction of depression that is characterized by an at least two point decline in total Hamilton Depression Rating Scale (HAM-D) value.
18. The method of embodiment 17, wherein after said administering for a period of at least 1 week, the patient experiences a reduction of depression that is characterized by an at least 50% reduction in HAM-D value.
19. The method of embodiment 17, wherein after said administering for a period of at least 1 week, the patient experiences a reduction of depression that is characterized by an at least one category change in HAM-D severity classification.
20. The method of embodiment 17, wherein after said administering for a period of at least 1 week, the patient experiences a reduction of depression that is characterized by HAM-D remission.
21. The method of any one of embodiments 1-16, wherein after said administering for a period of at least 1 week, the patient experiences a reduction of depression that is characterized by an at least two point decline in Montgomery Asberg Depression Rating Scale (MADRS) value.
22. The method of embodiment 21, wherein after said administering for a period of at least 1 week, the patient experiences a reduction of depression that is characterized by an at least 50% reduction in MADRS value.
23. The method of embodiment 21, wherein after said administering for a period of at least 1 week, the patient experiences a reduction of depression that is characterized by MADRS remission.
24. The method of any one of embodiments 1-16, wherein after said administering for a period of at least 1 week, the patient experiences a reduction of anxiety that is characterized by an at least two point decline in total Hamilton Rating Scale for anxiety (HAM-A) value.
25. The method of embodiment 24, wherein after said administering for a period of at least 1 week, the patient experiences a reduction of anxiety that is characterized by an at least 50% reduction in HAM-A value.
26. The method of embodiment 24, wherein after said administering for a period of at least 1 week, the patient experiences a reduction of anxiety that is characterized by an at least one category change in HAM-A severity classification.
27. The method of any one of embodiments 1-16, wherein after said administering for a period of at least 1 week, the patient experiences a reduction of depression that is characterized by at least one point decline in one or more of the Clinical Global Impression (CGI) subscale scores, wherein the CGI subscales are selected from Severity of Illness Subscale (CGI-S) or Global Improvement Subscale (CGI-I).
28. The method of any one of embodiments 1-16, wherein after said administering for a period of at least 1 week, the patient experiences a reduction of depression that is characterized by at least about a 10%, 20%, or 30% improvement in Symptoms of Depression Questionnaire (SDQ) total scale score or in any of the respective subscales of SDQ-1, SDQ-2, SDQ-3, SDQ-4 and SDQ-5.
29. The method of any one of embodiments 1-16, wherein after said administering for a period of at least 1 week, the patient experiences a reduction of depression that is characterized by an at least one point decline in Pittsburgh Sleep Quality Index (PSQI) Global score.
30. The method of any one of embodiments 1-29, wherein the patient is an MDD patient with insomnia.
31. The method of embodiment 30, wherein after said administering for a period of at least 1 week, the patient experiences a substantial reduction in insomnia compared to prior to said administering.
32. The method of embodiment 31, wherein after said administering for a period of at least 1 week, the patient experiences a reduction of insomnia that is characterized by at least about a 30% decline in wake time after sleep onset (WASO) compared to prior to the treatment.
33. The method of embodiment 31, wherein after said administering for a period of at least 1 week, the patient experiences a reduction of insomnia that is characterized by at least about a 30% increase in Total Sleep Time (TST) compared to prior to the treatment.
34. The method of embodiment 31, wherein after said administering for a period of at least 1 week, the patient experiences a reduction of insomnia that is characterized by at least about a 30% increase in sleep efficiency (SE) compared to prior to the treatment.
35. The method of embodiment 31, wherein after said administering for a period of at least 1 week, the patient experiences a reduction of insomnia that is characterized by at least about a 30% decrease in latency to persistent sleep (LPS) compared to prior to the treatment.
36. The method of embodiment 31, wherein after said administering for a period of at least 1 week, the patient experiences a reduction of insomnia characterized by at least a one point decline in Global Pittsburgh Sleep Quality Index (PSQI) score compared to prior to the treatment.
37. The method of embodiment 31, wherein after said administering for a period of at least 1 week, the patient experiences a reduction of insomnia that is characterized by at least a one point increase in Epworth Sleepiness Scale value compared to prior to the treatment.
38. The method of embodiment 31, wherein after said administering for a period of at least 1 week, the patient experiences a reduction of insomnia that is characterized by at least a one point decrease in Insomnia Severity Index scale value compared to prior to the treatment.
39. The method of embodiment 31, wherein after said administering for a period of at least 1 week, the patient experiences a reduction of insomnia that is characterized by at least about a 10% improvement in total Leeds Sleep Evaluation Questionnaire value compared to prior to the treatment.
40. The method of embodiment 31, wherein after said administering for a period of at least 1 week, the patient experiences a reduction of insomnia that is characterized by at least a one point decrease in total Athens Insomnia Scale value compared to prior to the treatment.
41. The method of embodiment 31, wherein after said administering for a period of at least 1 week, the patient experiences a reduction of insomnia that is characterized by a one point decrease in total Sleep Quality Index value compared to prior to the treatment.
42. The method of any one of embodiments 1-41, wherein Compound 1 is a pharmaceutically acceptable salt.
43. The method of any one of embodiments 1-42, wherein the pharmaceutically acceptable salt is selected from the group consisting of hydrobromide, citrate, malate, mesylate, phosphate, and tartrate.
44. The method of any one of embodiments 1-43, wherein the administration to a patient in need thereof provides a mean steady state blood plasma AUC (0-24) hours from about 500 to about 2500 ng*h/ml of the Compound 1.
45. The method of any one of embodiments 1-43, wherein the administration to a patient in need thereof provides a mean steady state blood plasma Cₘₐₓ from about 25 ng/mL to about 600 ng/ml of the Compound 1.
46. The method of any one of embodiments 1-43, wherein the administration to a patient in need thereof provides a mean steady state blood plasma Cₘₐₓ that does not exceed 600 ng/ml of the Compound 1.
47. The composition of any one of embodiments 1-46, wherein the composition is an oral dosage form.
48. The method of any one of embodiments 1-47, wherein Compound 1 is in the form of an extended release oral dosage form.
49. The method of any one of embodiments 1-48, further comprising administering one or more additional antidepressants.
50. The method of embodiment 49, wherein the additional antidepressant is selected from the group consisting of selective serotonin reuptake inhibitors, serotonin norepinephrine reuptake inhibitors, tricyclic antidepressants, monoamine oxidase inhibitors, mirtazapine bupropion, lamotrigine and atypical antipsychotics.
51. The method of embodiment 50, wherein the selective serotonin reuptake inhibitor is selected from the group consisting of fluoxetine, escitalopram, citalopram, sertraline, and paroxetine.
52. The method of embodiment 50, wherein the serotonin norepinephrine reuptake inhibitor is selected from the group consisting of venlafaxine and duloxetine.
53. The method of embodiment 50, wherein the serotonin tricyclic antidepressant is selected from the group consisting of amitriptyline, imipramine, and nortriptyline.
54. The method of embodiment 50, wherein the monoamine oxidase inhibitor is selected from the group consisting of phenelzine and tranylcypromine.
55. The method of embodiment 50, wherein the atypical antipsychotic is selected from the group consisting of lurasidone, aripiprazole, risperidone, olanzapine, quetiapine, ziprasidone, clozapine, iloperidone, paliperidone, asenapine and olanzapine/fluoxetine.
56. A method of treating a mood or affective disorder in a patient in need thereof comprising orally administering a daily dose of about 5 mg to about 120 mg of Compound 1:

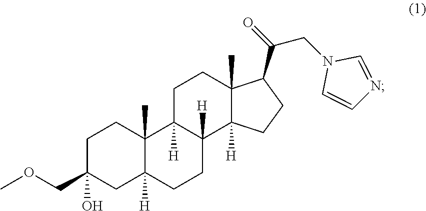

or a pharmaceutically acceptable salt thereof, wherein the mood or affective disorder is selected from the group consisting of perimenopause, menopause, generalized anxiety disorder, panic disorder, social anxiety disorder, acute stress disorder, post-traumatic stress disorder, specific phobia, and selective mutism.
57. The method of embodiment 56, wherein the mood or affective disorder is acute stress disorder.
58. The method of embodiment 56, wherein the mood or affective disorder is post-traumatic stress disorder.
59. The method of any one of embodiments 1-6 and 14-58, wherein about 20 mg of Compound 1 or a pharmaceutically acceptable salt thereof is administered once a day.
60. The method of any one of embodiments 1-6 and 14-58, wherein about 20 mg of Compound 1 or a pharmaceutically acceptable salt thereof is administered twice a day.
61. The method of any one of embodiments 1-6 and 14-58, wherein about 25 mg of Compound 1 or a pharmaceutically acceptable salt thereof is administered once a day.
62. The method of any one of embodiments 1-6 and 14-58, wherein about 25 mg of Compound 1 or a pharmaceutically acceptable salt thereof is administered twice a day.
63. The method of any one of embodiments 1-6 and 14-58, wherein about 35 mg of Compound 1 or a pharmaceutically acceptable salt thereof is administered once a day.
64. The method of any one of embodiments 1-6 and 14-58, wherein about 40 mg of Compound 1 or a pharmaceutically acceptable salt thereof is administered once a day.
65. The method of any one of embodiments 1-6 and 14-58, wherein about 45 mg of Compound 1 or a pharmaceutically acceptable salt thereof is administered once a day.
66. The method of any one of embodiments 1-6 and 14-58, wherein about 50 mg of Compound 1 or a pharmaceutically acceptable salt thereof is administered once a day.
67. The method of any one of embodiments 1-6 and 14-58, wherein about 55 mg of Compound 1 or a pharmaceutically acceptable salt thereof is administered once a day.
68. The method of any one of embodiments 1-6 and 14-58, wherein about 60 mg of Compound 1 or a pharmaceutically acceptable salt thereof is administered once a day.
69. The method of any one of embodiments 1-6 and 14-58, wherein about 65 mg of Compound 1 or a pharmaceutically acceptable salt thereof is administered once a day.
70. The method of any one of embodiments 1-6 and 14-58, wherein about 70 mg of Compound 1 or a pharmaceutically acceptable salt thereof is administered once a day.
71. The method of any one of embodiments 1-6 and 14-58, wherein about 75 mg of Compound 1 or a pharmaceutically acceptable salt thereof is administered once a day.
72. The method of any one of embodiments 1-6 and 14-58, wherein about 80 mg of Compound 1 or a pharmaceutically acceptable salt thereof is administered once a day.
73. The method of any one of embodiments 1-6 and 14-58, wherein about 85 mg of Compound 1 or a pharmaceutically acceptable salt thereof is administered once a day.
74. The method of any one of embodiments 1-6 and 14-58, wherein about 90 mg of Compound 1 or a pharmaceutically acceptable salt thereof is administered once a day.
75. The method of any one of embodiments 1-6 and 14-58, wherein about 95 mg of Compound 1 or a pharmaceutically acceptable salt thereof is administered once a day.
76. The method of any one of embodiments 1-6 and 14-58, wherein about 100 mg of Compound 1 or a pharmaceutically acceptable salt thereof is administered once a day.
76(a) The method of any one of embodiments 1-6 and 14-58, wherein about 15 mg of Compound 1 or a pharmaceutically acceptable salt thereof is administered once a day.
77. A method of treating a substance addiction disorder comprising administering to a patient in need thereof a therapeutically effective amount of Compound 1 or a pharmaceutically acceptable salt thereof
78. The method of claim 77, wherein the substance addiction disorder is an opioid use disorder.
79. The method of claim 77, wherein the substance addiction disorder is a cocaine use disorder.
80. The method of claim 77, wherein the substance addiction disorder is alcohol use disorder.
81. The method of claim 77, wherein the substance addiction disorder is benzodiazepine use disorder.
82. The method of any one of claims 78-81, wherein after the administering, the patient experiences a substantial reduction in substance addiction disorder compared to prior to said administering.
83. The method of claim 78, wherein after the administering, the patient experiences a reduction of opioid use disorder that is characterized by abstinence to opioid use during the period of Compound 1 administration.
84. The method of any one of claims 78 and 83, wherein after the administering, the patient experiences a reduction of opioid use disorder that is characterized by a statistically significant decrease in the percentage of opioid-free weeks in the Compound 1 treated group compared to a placebo treated group during the period of Compound 1 administration.
85. The method of any one of claims 78 and 83-84, wherein after the administering, the patient experiences a reduction of opioid use disorder that is characterized by substantial improvement in craving assessment compared to prior to the treatment.
86. The method of any one of claims 78 and 83-85, wherein after the administering, the patient experiences a reduction of opioid use disorder that is characterized by a statistically significant change in retention assessment compared to a placebo treated group.
87. The method of any one of claims 78 and 83-86, wherein after the administering, the patient experiences a reduction of opioid use disorder that is characterized by a significant statistical difference in the mean change in number of days of retention in Compound 1 treatment group relative to placebo.
88. The method of any one of claims 78-87, wherein the Compound 1 is administered as an adjunctive to methadone; buprenorphine; buprenorphine and naloxone; naltrexone, benzodiazepine, lofexidine, medically supervised withdrawal (detoxification), residential rehabilitation treatment, mutual help groups or outpatient substance use disorder services (e.g., counseling or medication for addiction), or combinations thereof
89. A method of treating major depressive disorder (MDD) in a patient in need thereof comprising orally administering a daily dose of from about 30 mg to about 120 mg of Compound 1:

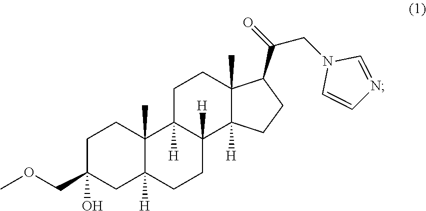

or a pharmaceutically acceptable salt thereof to a patient in need thereof to treat MDD.
90. The method of embodiment 1, wherein prior to said treatment, the patient's total Hamilton Depression Rating Scale (HAM-D) value is at least 22.
91. The method of any one of embodiments 89-90, wherein about 45 mg to about 80 mg of Compound 1 or a pharmaceutically acceptable salt thereof is administered.
92. The method of any one of embodiments 89-90, wherein about 45 mg of Compound 1 or a pharmaceutically acceptable salt thereof is administered.
93. The method of embodiment 90, wherein about 60 mg of Compound 1 or a pharmaceutically acceptable salt thereof is administered.
94. The method of any one of embodiments 89-90, wherein about 80 mg of Compound 1 or a pharmaceutically acceptable salt thereof is administered.
95. The method of any one of embodiments 89-94, wherein Compound 1 or a pharmaceutically acceptable salt thereof is administered once daily.
96. The method of any one of embodiments 89-95, wherein Compound 1 or a pharmaceutically acceptable salt thereof is administered at bedtime.
97. The method of any one of embodiments 89-96, wherein Compound 1 or a pharmaceutically acceptable salt thereof is administered without regard to meals.
98. The method of any one of embodiments 89-97, wherein the method comprises administering Compound 1 or a pharmaceutically acceptable salt thereof for about 1 week, about 2 weeks, about 3 weeks, about 4 weeks, about two months, about three months, about four months, about five months, about six months, about seven months, about eight months, about nine months, about ten months, about eleven months, about twelve months, about 18 months, about 24 months, about 30 months or about 36 months.
99. The method of any one of embodiments 89-98, wherein the method comprises continuous administration of Compound 1 or a pharmaceutically acceptable salt thereof
100. The method of embodiment 99, wherein the method comprises:
   (a) administering Compound 1 or a pharmaceutically acceptable salt thereof for about 1 week and
   (b) after the administration period (a) not administering Compound 1 or a pharmaceutically acceptable salt thereof for at least 3 weeks.
101. The method of embodiment 99, wherein the method comprises:
   (a) administering Compound 1 or a pharmaceutically acceptable salt thereof for about 3 weeks and
   (b) after the administration period (a) not administering Compound 1 or a pharmaceutically acceptable salt thereof for at least 3 weeks.
102. The method of embodiment 99, wherein the method comprises:
   (a) administering Compound 1 or a pharmaceutically acceptable salt thereof for about 4 weeks and
   (b) after the administration period (a) not administering Compound 1 or a pharmaceutically acceptable salt thereof for at least 3 weeks.
103. The method of any one of embodiments 89-98, wherein the method comprises intermittent administration of Compound 1 or a pharmaceutically acceptable salt thereof
104. The method of embodiment 103, intermittent administration comprises:
   (a) administering Compound 1 or a pharmaceutically acceptable salt thereof for a first administration period;
   (b) after the first administration period (a), not administering Compound 1 or a pharmaceutically acceptable salt thereof for a cessation period;
   (c) after the cessation period (b), administering Compound 1 or a pharmaceutically acceptable salt thereof for a second administration period.
105. The method of embodiment 103, wherein the intermittent administration comprises:
   (a) administering Compound 1 or a pharmaceutically acceptable salt thereof for about 1 week;
   (b) after the administration period (a) not administering Compound 1 or a pharmaceutically acceptable salt thereof for about 1 week; and
   (c) after the cessation period (b) administering Compound 1 or a pharmaceutically acceptable salt thereof for about 1 week.
106. The method of embodiment 103, wherein the intermittent administration comprises:
   (a) administering Compound 1 or a pharmaceutically acceptable salt thereof for about 2 weeks;
   (b) after the administration period (a) not administering Compound 1 or a pharmaceutically acceptable salt thereof for about 2 weeks; and
   (c) after the cessation period (b) administering Compound 1 or a pharmaceutically acceptable salt thereof for about 2 weeks.
107. The method of any one of embodiments 103-106, further comprising administering Compound 1 or a pharmaceutically acceptable salt thereof for one or more additional cessation periods.
108. The method of any one of embodiments 103-107, further comprising administering Compound 1 or a pharmaceutically acceptable salt thereof for one or more additional administration periods.
109. The method of any one of embodiments 103-104 and 107-108, wherein the first administration period is about one week, about two weeks, about three weeks, about four weeks, about five weeks, about six weeks, about seven weeks, or about eight weeks.
110. The method of any one of embodiments 103-104 and 107-109, wherein the cessation period is about one week, about two weeks, about three weeks, about four weeks, about five weeks, about six weeks, about seven weeks, or about eight weeks.
111. The method of any one of embodiments 103-104 and 107-110, wherein, the second administration period is about one week, about two weeks, about three weeks, about four weeks, about five weeks, about six weeks, about seven weeks, or about eight weeks.
112. The method of any one of embodiments 103-104 and 107-111, wherein the first administration period is about one week; the cessation period is about three weeks; and the second administration period is about one week.
113. The method of any one of embodiments 103-104 and 107-111, wherein the first administration period is about two weeks; the first cessation period is about two weeks; the second administration period is about one week; the second cessation period is about one week and the third administration period is about one week.
114. The method of any one of embodiments 103-104 and 107-111, wherein intermittent administration period is about one month, about two months, about three months, about four months, about five months, about six months, about seven months, about eight months, about nine months, about ten months, about eleven months, about twelve months, about 18 months, about 24 months, about 30 months or about 36 months.
115. The method of any one of embodiments 89-114, further comprising titrating the dose of Compound 1 or a pharmaceutically acceptable salt thereof for at least one week until a maintenance dose is achieved in the patient.
116. The method of embodiment 115, wherein the initial dose of Compound 1 or a pharmaceutically acceptable salt thereof is from about 15 mg to about 45 mg.
117. The method of any one of embodiments 115-116, wherein the maintenance dose of Compound 1 or a pharmaceutically acceptable salt thereof is from about 45 mg to about 80 mg.
118. The method of any one of embodiments 115-117, wherein the initial dose is administered for one week and the maintenance dose is administered for at least one week.
119. The method of any of embodiments 89-98, wherein the method comprises:
   (a) administering a loading dose of Compound 1 or a pharmaceutically acceptable salt thereof to a patient in need thereof and
   (b) administering a maintenance dose of Compound 1 or a pharmaceutically acceptable salt thereof
120. The method of embodiment 119, wherein the loading dose is administered for about 1 day, about 2 days, about 3 days, about 4 days, about 5 days, about 6 days, about 7 days, about 8 days, about 9 days, about 10 days, about 11 days, about 12 days, about 13 days or about 14 days.
121. The method of embodiment of any one of embodiments 119-120, wherein the loading dose of Compound 1 or a pharmaceutically acceptable salt thereof is about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg.
122. The method of embodiment of any one of embodiments 115 and 119-121, wherein the maintenance dose is administered for about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, or about 36 months.
123. The method of embodiment of any one of embodiments 115 and 119-122, wherein the maintenance dose of Compound 1 or a pharmaceutically acceptable salt thereof is about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg.
124. The method of embodiment of any one of embodiments 119-123, wherein the method further comprises a cessation period after administration of the loading dose and prior to administration of the maintenance dose.
125. The method of embodiment 124, wherein the cessation period is about one day, about two days, about three days, about four days, about five days, about six days, or about seven days.
126. The method of embodiment 124, wherein the cessation period is about one week, about two weeks, about three weeks, about four weeks, about five weeks, about six weeks, about seven weeks, or about eight weeks.
127. The method of any one of embodiments 89-126, wherein the administering provides a mean steady state AUG₀₋₂₄ of from about 600 ng·h/mL to about 900 ng·h.
128. The method of any one of embodiments 89-127, wherein the administering provides a mean steady state Cmax of from about 25 ng/mL to about 600 ng/mL.
129. The method of any one of embodiments 89-128, wherein the administering provides a mean steady state Cmax of from about 125 ng/mL to about 250 ng/mL.
130. The method of any one of embodiments 89-129, wherein after the administering, the patient experiences a substantial reduction in depression compared to prior to said administering.
131. The method of any one of embodiments 89-130, wherein after the administering, the patient experiences a reduction of depression that is characterized by an at least ten point decline in total Hamilton Depression Rating Scale (HAM-D) value.
132. The method of embodiment 131, wherein after the administering, the patient experiences a reduction of depression that is characterized by an at least 50% reduction in HAM-D value.
133. The method of embodiment 131, wherein after the administering, the patient experiences a reduction of depression that is characterized by an at least one category change in HAM-D severity classification.
134. The method of embodiment 131, wherein after the administering, the patient experiences a reduction of depression that is characterized by HAM-D remission.
135. The method of any one of embodiments 89-134, wherein after the administering, the patient experiences a reduction of depression that is characterized by an at least two point decline in Montgomery Asberg Depression Rating Scale (MADRS) value.
136. The method any one of embodiments 89-135, wherein after the administering, the patient experiences a reduction of depression that is characterized by an at least 50% reduction in MADRS value.
137. The method any one of embodiments 89-136, wherein after the administering, the patient experiences a reduction of depression that is characterized by MADRS remission.
138. The method of any one of embodiments 89-137, wherein after the administering, the patient experiences a reduction of depression that is characterized by at least one point decline in one or more of the Clinical Global Impression (CGI) subscale scores, wherein the CGI subscales are selected from Severity of Illness Subscale (CGI-S) or Global Improvement Subscale (CGI-I).
139. The method of any one of embodiments 89-138, wherein after the administering, the patient experiences a reduction of depression that is characterized by at least about a 10%, 20%, or 30% improvement in Symptoms of Depression Questionnaire (SDQ) total scale score or in any of the respective subscales of SDQ-1, SDQ-90, SDQ-3, SDQ-4 and SDQ-5.
140. The method of any one of embodiments 89-139, wherein after the administering, the patient experiences a reduction of depression that is characterized by an at least one point decline in Pittsburgh Sleep Quality Index (PSQI) Global score.
141. The method of any one of embodiments 89-140, wherein the patient is an MDD patient with insomnia.
142. The method of embodiment 141, wherein after the administering, the patient experiences a substantial reduction in insomnia compared to prior to said administering.
143. The method of embodiment 142, wherein after the administering, the patient experiences a reduction of insomnia that is characterized by at least about a 30% decline in wake time after sleep onset (WASO) compared to prior to the treatment.
144. The method of embodiment 142, wherein after the administering, the patient experiences a reduction of insomnia that is characterized by at least about a 30% increase in Total Sleep Time (TST) compared to prior to the treatment.
145. The method of embodiment 142, wherein after the administering, the patient experiences a reduction of insomnia that is characterized by at least about a 30% increase in sleep efficiency (SE) compared to prior to the treatment.
146. The method of embodiment 142, wherein after the administering, the patient experiences a reduction of insomnia that is characterized by at least about a 30% decrease in latency to persistent sleep (LPS) compared to prior to the treatment.
147. The method of embodiment 142, wherein after the administering, the patient experiences a reduction of insomnia characterized by at least a one point decline in Global Pittsburgh Sleep Quality Index (PSQI) score compared to prior to the treatment.
148. The method of embodiment 142, wherein after the administering, the patient experiences a reduction of insomnia that is characterized by at least a one point increase in Epworth Sleepiness Scale value compared to prior to the treatment.
149. The method of embodiment 142, wherein after the administering, the patient experiences a reduction of insomnia that is characterized by at least a one point decrease in Insomnia Severity Index scale value compared to prior to the treatment.
150. The method of embodiment 142, wherein after the administering, the patient experiences a reduction of insomnia that is characterized by at least about a 10% improvement in total Leeds Sleep Evaluation Questionnaire value compared to prior to the treatment.
151. The method of embodiment 142, wherein after the administering, the patient experiences a reduction of insomnia that is characterized by at least a one point decrease in total Athens Insomnia Scale value compared to prior to the treatment.
152. The method of embodiment 142, wherein after the administering, the patient experiences a reduction of insomnia that is characterized by a one point decrease in total Sleep Quality Index value compared to prior to the treatment.
153. The method of any one of embodiments 89-152, wherein Compound 1 is a pharmaceutically acceptable salt.
154. The method of embodiment 153, wherein the pharmaceutically acceptable salt is the citrate.

What is claimed is:

1. A method of treating major depressive disorder (MDD) in a patient in need thereof comprising orally administering a daily dose of from about 45 mg to about 80 mg of Compound 1: 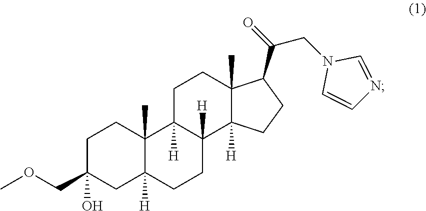
or a pharmaceutically acceptable salt thereof to a patient in need thereof to treat MDD.

2. The method of claim 1, wherein about 45 mg of Compound 1 or a pharmaceutically acceptable salt thereof is administered once a day.

3. (canceled)

4. The method of claim 1, wherein about 60 mg of Compound 1 or a pharmaceutically acceptable salt thereof is administered once a day.

5. (canceled)

6. The method of claim 1, wherein about 80 mg of Compound 1 or a pharmaceutically acceptable salt thereof is administered once a day.

7. (canceled)

8. (canceled)

9. The method of claim 1, wherein the method comprises administering Compound 1 or a pharmaceutically acceptable salt thereof for at least about 2 weeks.

10. The method of claim 1, wherein the method comprises administering Compound 1 or a pharmaceutically acceptable salt thereof for at least about 4 weeks.

11. The method of claim 1, wherein the administering provides a mean steady state Cₘₐₓ of from about 125 ng/mL to about 250 ng/mL.

12. The method of claim 1, wherein prior to said treatment, the patient's total Hamilton Depression Rating Scale (HAM-D) value is at least 22.

13. The method of claim 1, wherein after the administering, the patient experiences a reduction of depression that is at least 50% reduction in HAM-D value compared to prior to said treating.

14. The method of claim 1, wherein after the administering, the patient experiences a reduction of depression that is a decline in HAM-D value of about 6 points or greater compared to prior to said treating.

15. The method of claim 1, wherein after the administering, the patient experiences a reduction of depression that is HAM-D remission.

16. The method of claim 1, wherein after the administering, the patient experiences a reduction of depression that is at least a one category change in HAM-D severity classification compared to prior to said treating.

17. The method of claim 1, wherein after the administering, the patient experiences a reduction of depression that is at least 50% reduction in Montgomer-Asberg Depression Rating Scale (MADRS) value compared to prior to said treating.

18. The method of claim 1, wherein after the administering, the patient experiences a reduction of depression that is MADRS remission.

19. The method of claim 1, wherein the patient's MDD is resistant to treatment with other antidepressants.

20. The method of claim 1, wherein the Compound 1 or a pharmaceutically acceptable salt thereof is administered as an adjunctive antidepressant to existing antidepressant therapy.
